(19) United States
(12) Patent Application Publication
(10) Pub. No.: US 2011/0106106 A1
(43) Pub. Date: May 5, 2011

(54) TERMINATION DEVICES AND RELATED METHODS

(76) Inventors: Stephen C. Meier, Santa Clara, CA (US); Ann T. Meier, Santa Clara, CA (US)

(21) Appl. No.: 12/759,590

(22) Filed: Apr. 13, 2010

Related U.S. Application Data
(60) Provisional application No. 61/168,867, filed on Apr. 13, 2009.

(51) Int. Cl. A61B 17/04

(52) U.S. Cl. 606/139

(57) ABSTRACT

Devices, methods and kits for forming and/or advancing one or more knots in a tether (e.g., during a tissue modification procedure) are described. In some variations, a tether may be used to gather or compress tissue by bringing two pieces or sections of the tissue together. The tether, which may be under tension, may then be locked to maintain the tension (e.g., by forming one or more knots in the tether). In some variations, an open knot may be advanced along a tether before being converted into a closed knot. The devices, methods and/or kits may be used, for example, in minimally invasive procedures.

### CROSS-REFERENCE TO RELATED APPLICATIONS

This application claims the benefit of U.S. Provisional Application No. 61/168,867, filed on Apr. 13, 2009, the disclosure of which is incorporated herein by reference in its entirety.

### FIELD

The devices, methods and kits described herein relate generally to termination of tethers that have been deployed to a target site in a body of a subject. More specifically, the devices, methods and kits described herein relate to forming one or more knots in a tether after the tether has been deployed to a target site, and advancing the knot or knots distally, in the direction of the target site.

### BACKGROUND

Many different types of medical procedures involve the use of tethers. For example, tethers may be used to gather or compress tissue (e.g., by bringing two pieces or sections of tissue together). The tissue may be, for example, soft tissue, such as muscle tissue or fat tissue. As an example, in some procedures, anchors coupled to a tether are embedded in tissue, and the tether is then pulled upon to provide a cinching effect that gathers or compresses the tissue via the anchors. Examples of devices and methods for such procedures applied to heart valve repair are described, for example, in U.S. patent application Ser. Nos. 11/232,190 (published as US 2006/0190030 A1), 11/270,034 (published as US 2006/0122633 A1), and 11/583,627 (published as US 2008/0172035 A1), all of which are incorporated herein by reference in their entirety.

Some methods of gathering or compressing tissue include threading a tether through two or more tissue regions, applying tension to the tether, and tying off or knotting the tether to maintain the tension. Extra tether material may then be cut and removed. However, the manipulation required when knotting a tether can be difficult (e.g., because of restricted space). As a result, some methods of knotting a tether may be unduly complicated and/or time-consuming.

Accordingly, it would be desirable to provide methods and devices for forming a knot in a tether and/or advancing a knot along a tether to a target site in the body (e.g., at the end of a procedure involving the tether). It would further be desirable for such methods and devices to be relatively easy and efficient to use.

### SUMMARY

Described here are devices, methods and kits for locking tethers, such as tethers that have been used to gather or compress tissue (e.g., by pulling two or more regions of the tissue together). Generally, the devices, methods and kits described here may involve forming one or more knots in a first portion of a tether and/or advancing the knot or knots to a second, more distal portion of the tether. In some variations, the knot or knots may be formed outside of the body, and may then be advanced into the body and tightened. The devices, methods and kits described here may, for example, allow for a tether to be locked relatively quickly and easily, thereby minimizing procedure time.

Certain variations of the devices described here comprise knot pushers for advancing a knot in a tether. In some variations, a knot pusher may comprise an elongated member, a first coupling member (e.g., a wire) slidably coupled to the elongated member, a second coupling member (e.g., a wire) fixedly coupled to the elongated member (where at least a portion of the second coupling member extends distally of the elongated member), a first bearing, and a second bearing distal of the first bearing, where the first and second bearings are fixedly coupled to the first coupling member and slidably coupled to the second coupling member. In certain variations, the knot pusher may further comprise a cutting member.

The elongated member may comprise a first lumen, and the first coupling member may be slidably disposed within at least a portion of the first lumen. In some cases, the elongated member may comprise a second lumen, and the second coupling member may be fixedly disposed within a portion of the second lumen.

In some variations, each of the bearings may comprise a tapered portion. In some such variations, the bearings may be slidably coupled to the second coupling member at the tapered portions. The tapered portions of the bearings may each have an aperture therethrough, and the first coupling member may pass through the apertures.

The knot pusher may further comprise a tubular member having a proximal end, a distal end, and a lumen therethrough, and the elongated member may be disposed within the tubular member. The tubular member may comprise a wall portion having a first aperture therethrough. The first and second bearings may be disposed within the tubular member, and the first aperture may be positioned adjacent the first bearing. The knot pusher may further comprise a hypotube having a second aperture therethrough. The hypotube may be disposed within the tubular member, such that the second aperture is aligned with the first aperture.

Certain variations of methods described here may be used to tie and/or advance a knot in a tether coupled to body tissue. The tether may be directly or indirectly coupled to the body tissue. For example, the tether may be directly affixed to the tissue, or may be fixedly or slidably coupled to an anchor that is embedded in the tissue. The body tissue may, for example, be cardiac tissue (e.g., heart valve tissue, such as mitral valve tissue). In some variations, the knot may be tied using a knot pusher, such as a knot pusher comprising first and second bearings and a coupling member slidably coupled to the first and second bearings. Such a method may comprise winding a portion of the tether around the first and second bearings to form an open knot in the tether, advancing the knot pusher distally to advance the open knot distally, withdrawing the coupling member from the first and second bearings, and forming a closed knot from the open knot after the coupling member has been withdrawn from the first and second bearings.

In some variations, the closed knot may be formed from the open knot by pulling the tether. The first and second bearings may be disposed in a lumen of a sheath comprising a wall portion having an aperture therethrough, and the method may further comprise passing the tether through the aperture. The tether may be coupled to a plurality of anchors that are coupled to the body tissue. The method may comprise using a lasso to wind the portion of the tether around the first and second bearings to form the open knot in the tether.

### BRIEF DESCRIPTION OF THE DRAWINGS

FIGS. 1A and 1B illustrate the gathering or compression of tissue of a subject using a tether.

FIG. 2A is a side view in partial cross-section of a variation of a device for forming and/or advancing a knot in a tether, and FIG. 2B is a side view of the device of FIG. 2A.

FIG. 2C is a perspective view of a component of the device of FIG. 2A, and FIGS. 2D and 2E are side and top views, respectively, of the component of FIG. 2C.

FIGS. 2F-2K depict a variation of a method of using the device of FIG. 2A.

FIG. 2L is a front view of the device of FIG. 2A.

FIG. 2M depicts a variation of a knot configuration around a portion of the device of FIG. 2A.

FIG. 3A is a perspective view of another variation of a device for forming and/or advancing a knot in a tether, and FIGS. 3B and 3C depict a variation of a method of using the device of FIG. 3A.

FIGS. 4A and 4B are perspective views of an additional variation of a device for forming and/or advancing a knot in a tether.

FIG. 5A is a perspective view of a variation of a device for forming and/or advancing a knot in a tether, and FIGS. 5B and 5C depict a variation of a method of using the device of FIG. 5A.

FIGS. 6A and 6B depict variations of a device and method for forming and/or advancing a knot in a tether.

FIGS. 7A and 7B show additional variations of a device and method for forming and/or advancing a knot in a tether.

FIGS. 8A and 8B depict further variations of a device and method for forming and/or advancing a knot in a tether.

FIGS. 9A and 9B show variations of a device and method for forming and/or advancing a knot in a tether.

FIG. 10A depicts a variation of a knot configuration, and FIG. 10B depicts a variation of a device for advancing a knot in a tether.

FIG. 11A is a perspective view of a variation of a component of a device for advancing a knot in a tether, FIG. 11B is a side view of a variation of a device comprising the component of FIG. 11A, and FIG. 11C is an illustrative cross-sectional depiction of the component of FIG. 11A in use.

FIG. 12 is a perspective view of a variation of a device for advancing a knot in a tether.

FIG. 13 is a perspective view of a variation of a component of a device for advancing a knot in a tether.

FIGS. 14A and 14B are illustrative cross-sectional depictions of a variation of a component of a device for advancing a knot in a tether, and a variation of a related method.

FIG. 14C is an illustrative cross-sectional depiction of another variation of a component of a device for advancing a knot in a tether.

FIG. 15A is a perspective illustration of a variation of a device for advancing a knot in a tether, and FIG. 15B is a perspective view of a variation of a component of a device for advancing a knot in a tether.

FIGS. 16A and 16B are top and side views, respectively, of additional variations of devices for advancing a knot in a tether.

FIG. 17 is a perspective view of a variation of a device for forming and/or advancing a knot in a tether.

FIG. 18 is a perspective view of another variation of a device for forming and/or advancing a knot in a tether.

FIG. 19A is a top view of an additional variation of a device for advancing a knot in a tether, FIG. 19B is a side view of the device of FIG. 19A, and FIG. 19C is an illustrative top view of the device of FIG. 19A in use.

FIG. 20A is a side view of a variation of a device for advancing a knot in a tether, and FIG. 20B is a top view of a component of the device of FIG. 20A.

FIGS. 21A and 21B are perspective views of additional variations of devices for advancing a knot in a tether.

FIG. 22A is an illustration of a variation of a knot configuration, FIG. 22B provides another view of the knot configuration of FIG. 22A, and FIGS. 22C and 22D are side views of variations of devices and methods for advancing a knot in a tether.

FIG. 23A is a side cross-sectional view of a variation of a device for forming and/or advancing a knot in a tether, and FIG. 23B is a top cross-sectional view of a portion of the device of FIG. 23A.

FIG. 24A is a depiction of a variation of a knot configuration.

FIGS. 24B and 24C are side cross-sectional views of different variations of devices for advancing a knot in a tether.

FIG. 24D is a side view of a variation of a device for advancing a knot in a tether, and FIG. 24E is a perspective view of a variation of a device for advancing a knot in a tether.

FIG. 25A is a side view of a variation of a device for manipulating tissue, FIG. 25B is a front view of another variation of a device for manipulating tissue, FIGS. 25C-25F are side views of additional variations of devices for manipulating tissue, FIG. 25G is a front view of a further variation of a device for manipulating tissue, FIG. 25H is a side view of another variation of a device for manipulating tissue, and FIG. 25I is a front view of an additional variation of a device for manipulating tissue.

FIGS. 26A-26D are illustrations of different variations of tether-anchor assemblies comprising tethers having at least one knot formed therein.

FIG. 27 is a side view in partial cross-section of a variation of a device for cutting a tether.

### DETAILED DESCRIPTION

Described here are devices, methods and kits for locking a tether (e.g., after the tether has been tensioned to gather or compress tissue). In some variations, the devices, methods and kits described here may be used to form an open knot (or slip knot) in a more proximal portion of a tether and to advance the knot to a more distal portion of the tether, where the knot may then be tightened to lock the tether. Devices, methods and kits described here may be used both to form and advance a knot in a tether, or in some cases may be used just to form or advance a knot in a tether, as appropriate. The devices, methods and kits described here may allow for a knot to be formed outside of the body, and then to be advanced into a target site within the body. Additionally, the devices, methods and kits described here may allow for an open knot to be advanced to a target site without experiencing significant deformation, or even premature tightening, during advancement. Certain variations of the devices, methods and kits described here may be used to position or manipulate body tissue (e.g., heart valve leaflets) in a way that allows for greater ease and efficiency of tether knot formation and/or advancement. Tether-anchor configurations that may be used to tighten or compress tissue, such as heart valve tissue, are also described here.

The devices, methods and kits described here may be used in any appropriate procedure and location in which it is desired to form and/or advance one or more knots in a tether. While not so limited, the devices, methods and kits described here may be used, for example, in Natural Orifice Transluminal Endoscopic Surgery (“NOTES”) procedures, heart valve repair procedures (e.g., mitral valve annulus repair procedures), and/or endoscopic procedures (e.g., laparoscopy and/or arthroscopy). The devices, methods and kits described here may be used in non-invasive or minimally invasive procedures (e.g., minimally invasive percutaneous procedures), or in invasive procedures, such as invasive surgeries (e.g., open-heart surgeries), as appropriate. It should be understood that while devices, methods and kits described herein may be described with reference to heart tissue repair, they may be used in any appropriate instance involving the formation and/or advancement of knots in a medical procedure. Specific examples of devices, methods and kits will now be described in further detail below.

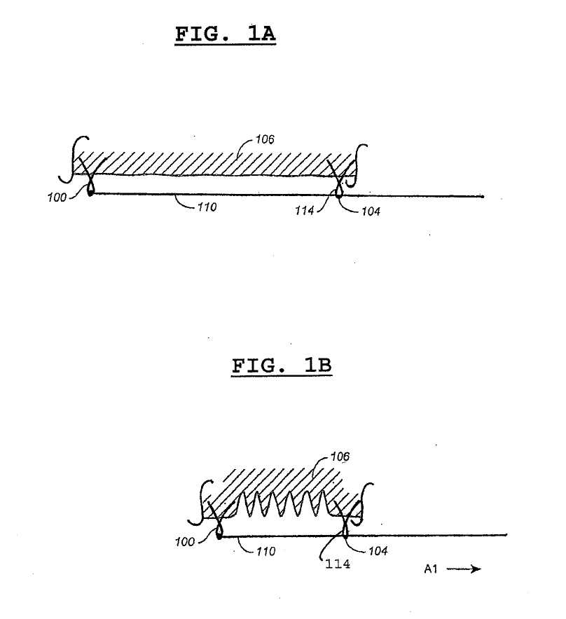

Turning now to the figures, FIG. 1A shows two anchors (100) and (104) anchored into tissue (106) of a subject. A tether (110) is fixedly attached to anchor (100), and is threaded through a loop region or eyelet (114) of anchor (104). As shown in FIG. 1B, when tether (110) is pulled upon in the direction of arrow (A1), a cinching effect results, such that anchors (100) and (104) are brought closer together, and the tissue length between anchors (100) and (104) is reduced. In this way, tissue (106) may be gathered and/or compressed. A tether may be manually pulled upon to provide this effect and/or one or more cinching devices may be applied to a tether to provide this effect. Non-limiting examples of cinching devices are described in U.S. patent application Ser. No. 12/576,955, filed on Oct. 9, 2009, which is hereby incorporated by reference in its entirety.

After tether (110) has been tensioned by a desired amount, one or more knots may be formed in tether (110), thereby locking the tether to maintain the tension in the tether. For example, tether (110) may be tensioned, and one or more knots may be formed in tether (110) while the tether is under tension. In some variations, the knot or knots may be located just proximal of a proximal-most anchor. For example, the knot or knots may be located just proximal of eyelet (114) of anchor (104). In some such variations, at least one of the knots (e.g., the knot closest to eyelet (114)) may be large enough to be prevented from passing through eyelet (114). As a result, the knot or knots can help to maintain the cinching effect provided by the tensioned tether. In some cases, after the knot or knots have been formed to secure the tension in tether (110), excess portions of the tether may thereafter be cut and removed (e.g., using one or more tether-cutting devices, such as those described in further detail below).

The above-described process may be used in a wide variety of tissues. For example, in some variations, anchors that are connected to each other by a tether may be deployed into tissue in the region of a mitral valve annulus. The tether may then be pulled upon to provide a cinching effect, which may restructure the mitral valve annulus (e.g., reducing mitral valve regurgitation). Thereafter, the tether may be locked in place by forming one or more knots in the tether, thereby maintaining the cinching effect. Finally, in some variations of methods, one or more cutting devices may be used to remove excess tether material. Mitral valve repair is described, for example, in U.S. patent application Ser. Nos. 11/232,190 (published as US 2006/0190030 A1), 11/270,034 (published as US 2006/0122633 A1), 11/583,627 (published as US 2008/0172035 A1), 11/656,141 (published as US 2008/0177380 A1), and 12/366,553 (published as US 2009/0222083 A1), all of which are hereby incorporated by reference in their entirety.

In certain variations, the above-described process may be used in a heart reshaping procedure, such as a ventricular remodeling procedure that is used to repair a heart experiencing valve dysfunction. Heart repair procedures, including heart reshaping procedures, are described, for example, in U.S. patent application Ser. No. 12/253,792 (published as US 2009/0234318 A1), which is hereby incorporated by reference in its entirety.

As discussed above, the devices, methods and kits described herein may be used, as appropriate, in any of a number of different sites within the body and/or to assist with any of a number of different types of procedures. As an example, the devices, methods and kits described herein may be used in NOTES procedures. As another example, the devices, methods and kits described herein may be used in heart procedures other than those involving mitral valve repair. For example, they may be used to repair an aortic valve or a tricuspid valve, or to secure a prosthetic heart valve, or they may be used in heart ports. As an additional example, the devices, methods and kits may be employed in a procedure in which one or more tethers are used to reinforce an annuloplasty ring. Furthermore, the devices, methods and kits described herein may be used, for example, in a variety of open surgical procedures.

Anchors for use with the devices, methods and kits described here may be any suitable anchor. The anchors may be made of any suitable material, may be any suitable size, and may be of any suitable shape. Additionally, while two anchors are shown in FIGS. 1A and 1B, in some cases multiple anchors may be used. In cases in which two or more anchors are used, the anchors may all have the same size and shape, or may have different sizes and/or shapes. The anchors may be made of one material or more than one material. Examples of anchor materials include super-elastic materials and/or shape memory materials, such as nickel-titanium alloys (e.g., Nitinol) and spring stainless steel. Examples of anchor shapes include T-tags, rivets, staples, hooks (e.g., C-shaped or semicircular hooks, curved hooks of other shapes, straight hooks, barbed hooks), multiple looped anchors, clips, and the like. The anchors may be configured to self-expand and self-secure into tissue, but need not be configured in such a fashion. Illustrative examples of suitable anchors are described in more detail, for example, in U.S. patent application Ser. Nos. 11/202,474 (published as US 2005/0273138 A1), 11/894,340 (published as US 2008/0058868 A1), 11/894,368 (published as US 2008/0045982 A1), 11/894,397 (published as US 2008/0045983 A1), 11/894,463 (published as US 2008/0051810 A1), and 11/894,468 (published as US 2008/0051832 A1), all of which are hereby incorporated by reference in their entirety. It should also be noted that while anchors have been described, any other type of suitable fasteners or implants (e.g., leads, electrodes, etc.) may be used with one or more of the devices, methods and/or kits described here. Additionally, some procedures employing the devices, methods and/or kits described here may not involve any anchors or other types of fasteners. As an example, certain variations of the devices, methods and kits described here may be used to lock a suture that has been sewn through tissue by forming and/or advancing one or more knots in the suture.

Tethers may be made from any suitable or desirable biocompatible material, and may be made of a single material or a combination of materials (e.g., a tether may be in the form of one long piece of material, or may comprise two or more pieces). Moreover, tethers may be braided or not braided, woven or not woven, and/or reinforced and/or impregnated with one or more additional materials. As non-limiting examples, a tether may be made from (1) one or more suture materials (e.g., absorbable suture materials such as polyglycolic acid and polydioxanone, natural fibers such as silk, and artificial fibers such as polypropylene, polyester, polyester impregnated with polytetrafluoroethylene, nylon, etc.), (2) one or more suture-like materials, (3) one or more metals (absorbable or non-absorbable), (4) one or more metal alloys (e.g., stainless steel), (5) one or more shape memory materials, such as shape memory alloys (e.g., a nickel titanium alloy), (6) one or more other biocompatible materials, or (7) any combination thereof. In some variations, a tether may be in the form of a DACRON® polyester strip. In certain variations, a tether may comprise high-density polyethylene (HDPE) or ultra-high molecular weight polyethylene (UHMWPE). In some variations, a tether may comprise polyetheretherketone (PEEK). Certain variations of tethers may have a braided textile construction. Some variations of tethers may be in the form of a wire. Additionally, a tether may include multiple layers, and/or may include one or more coatings. For example, a tether may be in the form of a polymer-coated wire. In certain variations, a tether may comprise a combination of one or more sutures and one or more wires. As an example, a tether may be formed of a suture that is braided with a wire. Some variations of tethers may be in the form of monofilament or multifilament textile yarns or fibers. In certain variations, a tether may be formed of one or more electrode materials. In some variations, a tether may be formed of one or more materials that provide for the telemetry of information (e.g., regarding the condition of the target site).

Some variations of tethers may include one or more therapeutic agents (e.g., drugs, such as time-release drugs). As an example, a tether may be partially or entirely coated with one or more therapeutic agents. In certain variations, a tether may be used to deliver one or more growth factors and/or genetic regenerative factors. In some variations, a tether may be coated with one or more materials (e.g., a polymer) that encapsulate or control the release rate of one or more therapeutic agents, or in which one or more therapeutic agents are embedded. The therapeutic agents may be used, for example, to treat a target site to which the tether is fixedly attached or otherwise secured. In certain variations, a tether may include one or more lumens through which one or more therapeutic agents may be delivered.

While a procedure for gathering or compressing tissue using one tether has been described, some variations of procedures for modifying tissue may involve the use of multiple tethers, such as 2, 3, 4, 5, or 10 tethers. In some cases in which multiple tethers are used, at least some of the tethers may be associated with (e.g., fixedly attached to) different anchors, and/or at least some of the tethers may be associated with (e.g., fixedly attached to) the same anchor. The devices, methods and kits described herein may be applied to a single tether, or to multiple tethers. As an example, a knot pusher may be used to form and/or advance a knot in more than one tether, either simultaneously, or at different times.

As described above, after one or more anchors have been secured and the tether has been tensioned, the tether may then be locked or secured into place to maintain the tension (and, therefore, the cinching effect). In some instances, one or more knots may be used to lock the tether. In certain variations, the knot or knots may be formed in a more proximal location of the tether (e.g., where it may be relatively easy to form the knot or knots, such as outside the body), and may then be pushed distally, to a different location of the tether (e.g., where it may be more difficult to form the knot or knots, such as a target site within the body). Different variations of knot pushers and knot-pushing methods are described in further detail below.

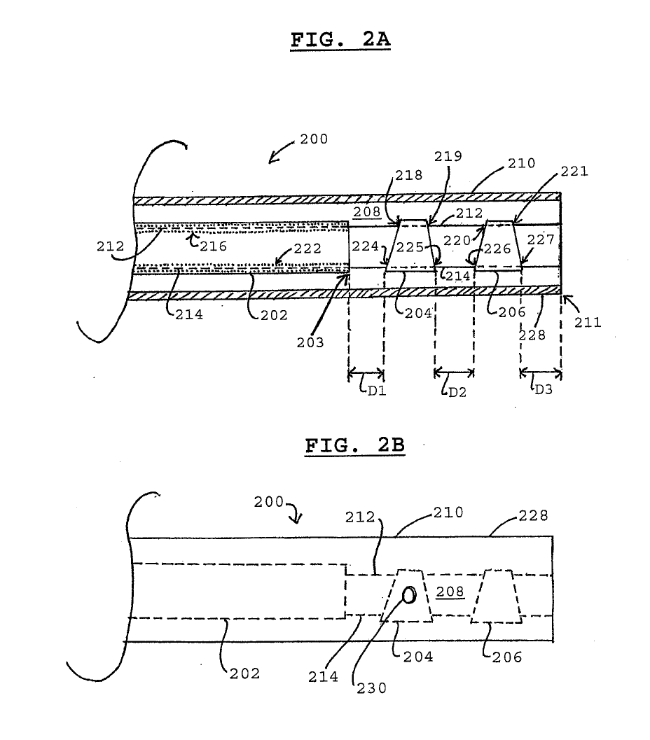

For example, FIG. 2A shows a knot pusher (200) comprising an elongated member (202) and two tapered bearings (204) and (206) disposed within a lumen (208) of a sheath (210). Elongated member (202) and/or sheath (210) may be formed of, for example, one or more polymers, such as PEBAX® polyether block amide (e.g., PEBAX® 4033 polymer or PEBAX® 5533 polymer), and/or one or more metal alloys. Sheath (210) may optionally comprise one or more braided regions (e.g., to provide support to the sheath). Sheath (210) may be sized according to the procedure in which it is to be used, as well as to accommodate elongated member (202) and bearings (204) and (206). In certain variations, sheath (210) may have an inner diameter of about 0.125 inch and/or an outer diameter of about 0.14 inch to about 0.15 inch. Sheath (210) may be configured to fit over a portion of elongated member (202), or may be configured to fit over the entire elongated member.

Bearings (204) and (206) are coupled to elongated member (202) via coupling members (212) and (214). Coupling member (212) is slidably disposed within a lumen (216) of elongated member (202), and slidably passes through apertures (218) and (219) of bearing (204), as well as apertures (220) and (221) of bearing (206). Thus, coupling member (212) may be proximally withdrawn or distally advanced as desired. Coupling member (214), however, is fixedly coupled to elongated member (202) and to bearings (204) and (206). More specifically, a portion of coupling member (214) is disposed within a lumen (222) of elongated member (202), and is fixedly coupled to the elongated member within the lumen. Additionally, coupling member (214) passes through apertures (224) and (225) of bearing (204), as well as apertures (226) and (227) of bearing (206), and is fixedly coupled (e.g., welded or soldered) to the bearings at these locations.

Coupling members (212) and (214) may be in the form of, for example, wires, tethers, etc., and may comprise any appropriate material or materials, such as metals, metal alloys, and/or polymers. As an example, in some variations, one or both of coupling members (212) and (214) may be formed of stainless steel or polytetrafluoroethylene (PTFE)-coated stainless steel. Coupling members (212) and (214) may be made of the same materials or different materials, and may have the same dimensions (e.g., length or cross-sectional diameter) or different dimensions. For example, in some variations, coupling members (212) and (214) may each have a cross-sectional diameter of about 0.014 inch to about 0.018 inch (e.g., about 0.0145 inch, about 0.015 inch, or about 0.016 inch). Furthermore, while two coupling members are shown, different numbers of coupling members may be used. For example, in some variations, a knot pusher may comprise two slidable coupling members and one fixed coupling member.

Bearings (204) and (206) may be formed of, for example, one or more metals, metal alloys, and/or polymers. The bearings may be made of the same material or materials, or may comprise one or more different materials. Additionally, at least one of the bearings may be made of the same material or materials as at least one of the coupling members. Alternatively, the bearings and coupling members may be made of different materials. Bearings (204) and (206) may generally be solid, or may have one or more hollow regions.

Referring again to FIG. 2A, bearing (204) is located at a distance (D1) from the distal end (203) of elongated member (202), and is separated from bearing (206) by a distance (D2). Additionally, bearing (206) is located at a distance (D3) from the distal end (211) of sheath (210). The distances may all be the same, or at least two of the distances may be different from each other. For example, in some variations, distances (D1), (D2), and (D3) may each be about 0.1 inch. Distance (D2) between bearings (204) and (206) may be selected, for example, based on the size and configuration of the knot to be formed around the bearings.

As shown in FIG. 2B, which depicts the exterior of knot pusher (200) with its inner components shown in phantom, sheath (210) comprises a wall portion (228) including an aperture (230) therethrough. In some variations, wall portion (228) may comprise a hypotube in the area of aperture (230) (e.g., to provide reinforcement to aperture (230)). The region of wall portion (228) comprising the hypotube may have a length of, for example, about 0.278 inch. Alternatively or additionally, the region of wall portion (228) distal to the region comprising the hypotube may have a length of about 0.238 inch, and/or the region of wall portion (228) between the proximal end of the hypotube and distal end (203) of elongated member (202) may have a length of about 0.103 inch. The hypotube may be formed of, for example, one or more metals and/or metal alloys, such as stainless steel.

Aperture (230) is sized and configured for passage of a tether therethrough and in some variations, aperture (230) may be radiused (e.g., to enhance such tether passage). As shown, aperture (230) is positioned adjacent bearing (204); however, other suitable locations may also be used for the aperture. Moreover, while only one aperture is shown in wall portion (228) of sheath (210), in some variations, a sheath wall portion may comprise multiple apertures (e.g., two, three, four, five, etc.) therethrough. The apertures may have the same size and shape, or may have different sizes and/or shapes. As an example, a sheath wall portion may include multiple apertures that are each sized differently to accommodate tethers of different sizes. In certain variations, a wall portion of a sheath may not have any apertures. In such cases, a tether may, for example, be passed into the lumen of the sheath via the distal opening of the sheath, and may exit the sheath at its proximal opening.

As depicted in FIGS. 2A and 2B, bearings (204) and (206) have an identical size and shape. FIGS. 2C-2E show different views of bearing (204) in enhanced detail. As shown there, bearing (204) is tapered, such that it is wider at its base (232) than it is at its top (234). This configuration may help a knot to slide off of the bearing once the knot has been advanced to a target site, as described below. In some variations, base (232) may have a width (W1) of about 0.08 inch. In certain variations, top (234) may have a width (W2) of about 0.05 inch. While tapered bearings having a particular configuration have been shown, any appropriately configured tapered bearing may be used, and in some cases, bearings that are not tapered may be used. Additionally, while bearings (204) and (206) are depicted as having the same size and shape, certain variations of knot pushers may comprise bearings having different sizes and/or shapes. As an example, a knot pusher may comprise at least one cylindrical bearing. Moreover, a knot pusher may have just one bearing or more than two bearings, depending, for example, on the desired size and configuration of the knot to be formed.

FIGS. 2F-2K illustrate a variation of a method of using knot pusher (200) to form a knot in a tether. (In FIGS. 2F-2K, lumens (216) and (222) have been removed for clarity.) As shown in FIG. 2F, sheath (210) has been retracted proximally and/or elongated member (202) has been pushed distally, so that bearings (204) and (206), as well as a portion of coupling member (214), are positioned outside of sheath (210). Additionally, slidable coupling member (212) has been withdrawn so that it does not pass through either bearing (204) or bearing (206). This configuration may allow an open knot to be formed relatively easily around bearings (204) and (206). For example, and referring now to FIG. 2G, a proximal portion of a tether (236) may be wrapped around bearings (204) and (206) to form a loose or open knot (238). While open knot (238) is depicted as having a certain configuration, in some variations, a knot pusher, such as knot pusher (200), may be suitable for use with more than one different type of open knot configuration. Tether (236) may, for example, be attached at its distal end to tissue and/or to an anchor. During knot formation, knot pusher (200) may be located outside of the body, to allow a knot to be relatively easily formed around bearings (204) and (206).

Referring now to FIG. 2H, after open knot (238) has been formed around bearings (204) and (206), coupling member (212) may be slidably routed through bearings (204) and (206). Once coupling member (212) has been routed through the bearings, open knot (238) is effectively locked into place between coupling members (212) and (214). Thus, open knot (238) may be prevented from prematurely dissociating from knot pusher (200) (e.g., as open knot (238) is being advanced toward a target site).

As shown in FIG. 2I, after open knot (238) has been locked into place, the operator may route the proximal end of tether (236) into lumen (208) of sheath (210). Tether (236) may then be passed through aperture (230) (FIG. 2B) in wall portion (228). In some variations, the operator may use a lasso to engage the tether and to thread the tether into the lumen of the sheath and through the aperture (e.g., by pulling on the opposite end of the lasso). After tether (236) has been routed into sheath (210) and through aperture (230), sheath (210) may be advanced over bearings (204) and (206), so that open knot (238) is positioned within lumen (208). Alternatively or additionally, elongated member (202) may be proximally withdrawn, so that bearings (204) and (206) and open knot (238) are also proximally withdrawn, eventually entering lumen (208) of sheath (210). Once open knot (238) is disposed within lumen (208), knot pusher (200) may be advanced over tether (236) to a target site. As an example, in a cardiac tissue repair procedure involving tethered anchors, open knot (238) may be advanced to a location just proximal of the proximal-most anchor on the tether.

Referring now to FIG. 2K, once knot pusher (200) has reached the target site, coupling member (212) may be proximally withdrawn, thereby “unlocking” open knot (238). Tether (236) may then be tensioned (either for the first time or for an additional time) by, for example, pulling proximally on the tether. This tensioning may cause open knot (238) to ride up the tapered sides of bearings (204) and (206), coming off of the tops of the bearings and turning into a closed knot (240). In certain variations, one or more other elements may be added to the knot during and/or after closure of the knot (e.g., to enhance the locking capabilities of the knot). For example, adhesive, glue, and/or cement may be added to the knot.

The placement of bearings (204) and (206) within lumen (208) of sheath (210), as well as the location of apertures (224) and (225) in bearing (204) and apertures (226) and (227) in bearing (206), may be at least partially determined by the configuration of open knot (238), as well as the dimensions of tether (236). For example, and referring to FIG. 2L, bearing (206) is positioned within sheath (210) such that it is separated from the inner wall of sheath (210) by dimensions (D4), (D5), and (D6). Additionally, coupling members (212) and (214) are positioned within bearing (206) such that there is a distance (D7) between them. Dimensions (D4) and (D6) may be determined, for example, by the space occupied by tether (236) wrapping around bearing (206). Dimensions (D4) and (D6) may be the same as each other or may be different from each other. Dimension (D5) may be selected, for example, based on providing sufficient room to withdraw or advance sheath (210) over bearing (206). Finally, dimension (D7) may be selected, for example, to provide sufficient space for tether (236) to ride between coupling members (212) and (214) as knot pusher (200) is advanced to a target site.

Any appropriate configuration of an open knot may be formed around bearings (204) and (206). For example, FIG. 2M shows a top view of another variation of an open knot (250) formed from a tether (252). Open knot (250) is formed around bearings (204) and (206) in a pretzel configuration. Such a configuration, which positions bearings (204) and (206) within the two upper loops (254) and (256) of open knot (250), may help to limit the likelihood of tether (252) twisting as the knot is advanced to a target site. While certain variations of knots have been shown, any other suitable variations may be employed. Moreover, in some variations, a knot may not loop around all of the bearings in a knot pusher, and/or may form multiple loops around at least one bearing.

Knot pusher (200) is generally configured as a catheter-type device. Other variations of knot pushers or knot formation and/or advancement devices may also have such a configuration. Additionally, other suitable configurations may be used. Moreover, knot pusher (200) may be scaled up (e.g., for use in a surgical procedure) or down (e.g., for use in a minimally invasive procedure), depending, for example, on the requirements of the particular procedure in which the knot pusher is to be used. The same option to scale up or down may, of course, also apply to other knot pushers or devices described herein, as appropriate.

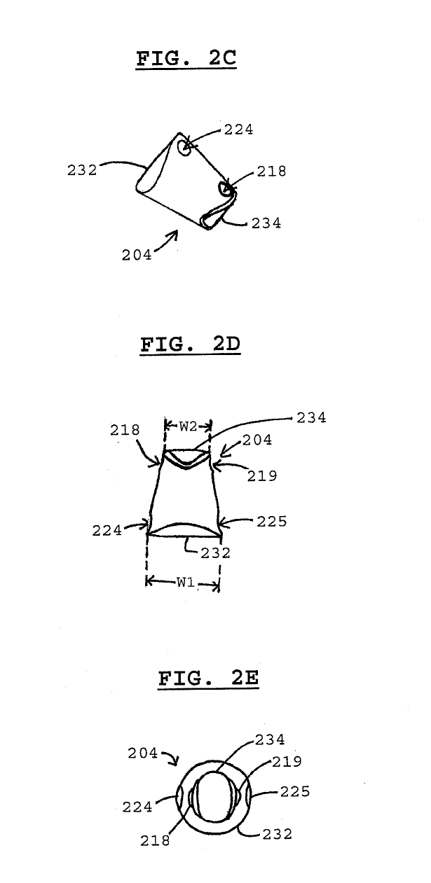

While one variation of a knot pusher has been shown, other variations may be used. For example, FIG. 3A shows a variation of a knot pusher having a clamping configuration similar to that of a tuning fork. As shown there, knot pusher (300) comprises a sheath (302) and a clamping member (304) capable of being withdrawn into, and advanced from, sheath (302). Clamping member (304) comprises clamping arms (306) and (308), each having a tapered post (310) or (312) in its distal portion. FIGS. 3B and 3C illustrate a method of using knot pusher (300). First, clamping member (304) is positioned sufficiently outside of sheath (302) to form an opening between posts (310) and (312). This allows a tether (314) to be wound around post (312) to form an open knot (316). Then, clamping member (304) may be withdrawn into sheath (302) and/or sheath (302) may be advanced over clamping member (304). As clamping arms (306) and (308) are compressed toward each other, posts (310) and (312) contact each other, thereby locking open knot (316) into place within knot pusher (300).

Next, tether (314) may be pulled through sheath (302), exiting at the proximal end of knot pusher (300). Knot pusher (300) may then be advanced over tether (314) (e.g., through a catheter) to a target site. While knot pusher (300) is advanced distally over tether (314), open knot (316) is forced to move along with knot pusher (300). Once knot pusher (300) has reached the target site, clamping member (304) may be pushed distally and/or sheath (302) may be withdrawn proximally. This allows clamping arms (306) and (308) to separate from each other, thereby releasing open knot (316). Tether (314) may then be tensioned to form a closed knot (318).

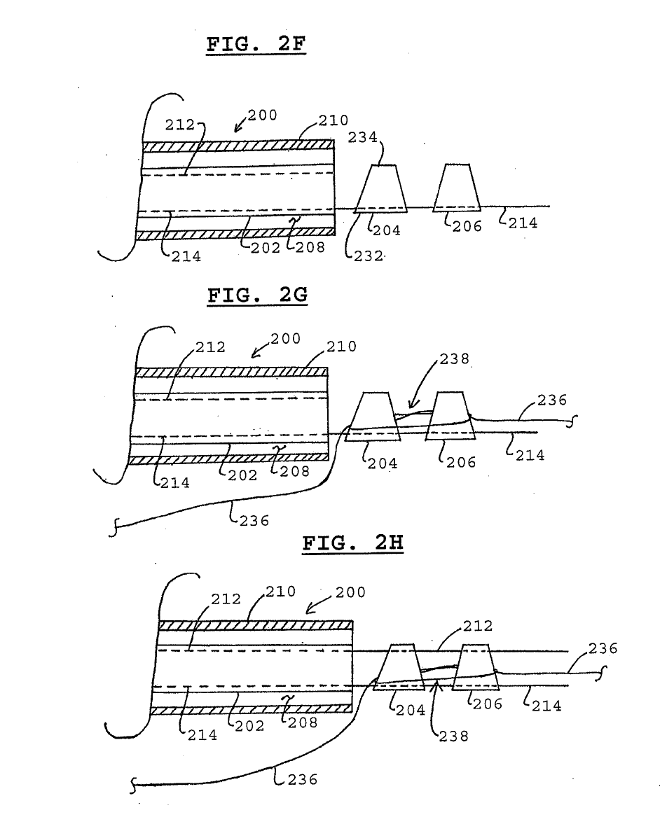

FIGS. 4A and 4B depict another variation of a knot pusher having a clamping configuration similar to that of a tuning fork. As shown there, knot pusher (400) comprises a sheath (402) and a clamping member (404) slidably disposed within sheath (402). Clamping member (404) comprises clamping arms (406) and (408), each having a post (410) or (412) in its distal portion. FIG. 4A shows knot pusher (400) when clamping member (404) has been withdrawn into sheath (402) and/or sheath (402) has been pushed over clamping member (404). In FIG. 4B, however, clamping member (404) has been partially pushed out of sheath (402) and/or sheath (402) has been partially withdrawn from clamping member (404). If clamping member (404) is pushed distally by an additional amount and/or sheath (402) is withdrawn proximally by an additional amount, then posts (410) and (412) may separate, thereby allowing a knot to be formed around one or both of the posts or to be released from one or both of the posts.

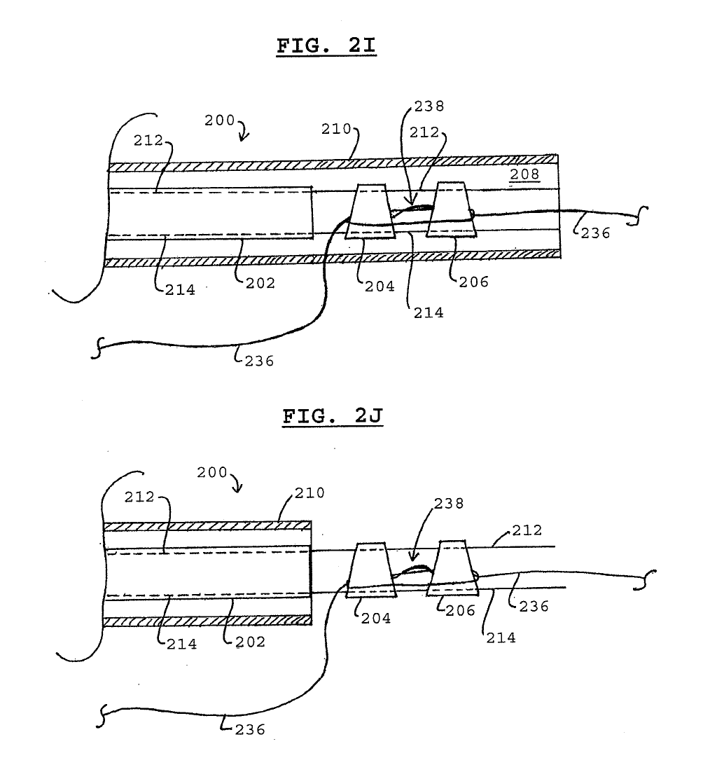

Other variations of clamping-type knot pushers may be used. As an example, FIG. 5A shows an additional variation of a knot pusher (500) having a clamping configuration similar to that of a tuning fork. As shown there, knot pusher (500) comprises a clamping member (502) including clamping arms (504) and (506). Knot pusher (500) also comprises a cylindrical wedge (508) disposed between clamping arms (504) and (506) and capable of being withdrawn or advanced via a push-pull wire (510). While a cylindrical wedge is shown, any appropriately shaped wedge may be used. For example, some variations of knot pushers may comprise a spherical, pyramidal, cubic, or irregularly shaped wedge. Additionally, in certain variations, a knot pusher may comprise multiple wedges. Push-pull wire (510) is routed through clamping member (502) and may be actuated, for example, by pulling or pushing on its proximal end (not shown). At its distal end (512), knot pusher (500) comprises two posts (514) and (516). Depending on the position of wedge (508), posts (514) and (516) may contact each other or may be separated from each other.

FIGS. 5B and 5C illustrate a method of using knot pusher (500). More specifically, FIG. 5B shows knot pusher (500) in a closed position, while FIG. 5C shows knot pusher (500) in an open position. In use, knot pusher (500) may first be placed in an open position, so that an open knot (518) may be formed by tying a tether (520) around post (514) and/or post (516). The knot pusher may be placed in an open position by, for example, pulling on push-pull wire (510) to move wedge (508) proximally. Clamping arms (504) and (506) are designed so that when wedge (508) is in a sufficiently proximal position, clamping arms (504) and (506) are forced apart, causing posts (514) and (516) to lose contact with each other.

Once open knot (518) has been formed, wedge (508) may be distally advanced, thereby allowing posts (514) and (516) to contact each other. In this closed position, shown in FIG. 5B, open knot (518) may effectively be locked into place and prevented from coming loose or undone. Open knot (518) may then be advanced to a target site by, for example, pushing knot pusher (500) to the target site via a catheter or sheath (not shown). Once at the target site, knot pusher (500) may be removed from the catheter or sheath (e.g., by withdrawing the catheter or sheath). Referring now to FIG. 5C, knot pusher (500) may then be manipulated into its open position by pulling on push-pull wire (510) until wedge (508) is sufficiently proximal to cause posts (514) and (516) to separate from each other. This allows open knot (518) to be released from its locked position. When tether (520) is pulled upon, open knot (518) may then be formed into a closed knot (522).

Knot pushers having clamping configurations similar to that of a tuning fork have been described. However, other variations of knot pushers having clamping configurations may be used to advance one or more knots to a target site. For example, in some variations, a knot pusher may comprise a clamping member having two separate articulatable clamping arms, or a clamping member having more than two clamping arms. Moreover, certain variations of knot pushers may not have clamping configurations.

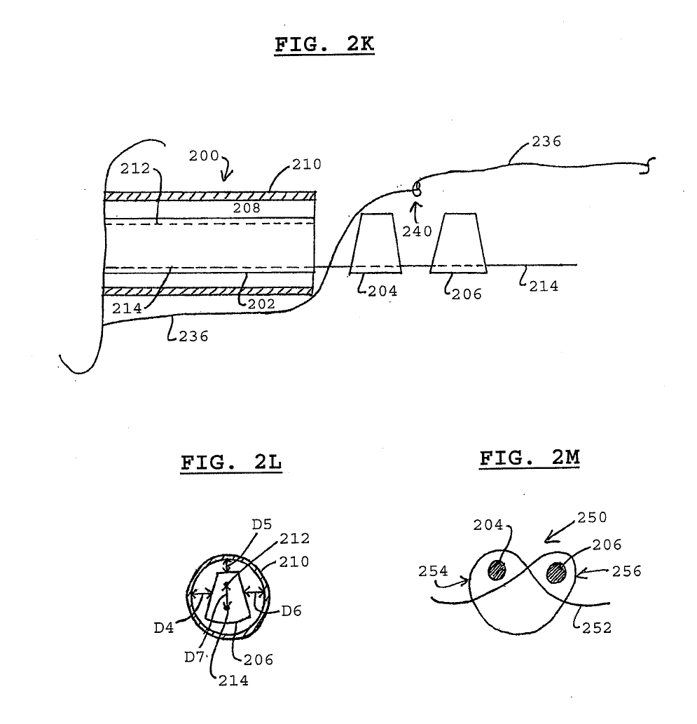

For example, FIGS. 6A and 6B depict a variation of a knot pusher that does not have a clamping configuration. Referring first to FIG. 6A, a knot pusher (600) comprises a first portion (602), a second portion (604), and a push-pull rod (606) that may be used to slide first portion (602) relative to second portion (604). First portion (602) has a distal region (608) comprising a post (610), and second portion (604) similarly has a distal region (612) comprising a post (614). FIG. 6A shows knot pusher (600) in its closed position, in which posts (610) and (614) contact each other. As shown in FIG. 6A, an open knot (616) that has been formed in a tether (618) is locked between first and second portions (602) and (604). During use, knot pusher (600) may be advanced to a target site (e.g., via a catheter or sheath). Once at the target site (and, e.g., once removed from the catheter or sheath), knot pusher (600) may be converted into its open position by pushing on push-pull rod (606), and thereby causing posts (610) and (614) to separate from each other (FIG. 6B). Tether (618) may then be pulled upon to form a closed knot (620) from open knot (616).

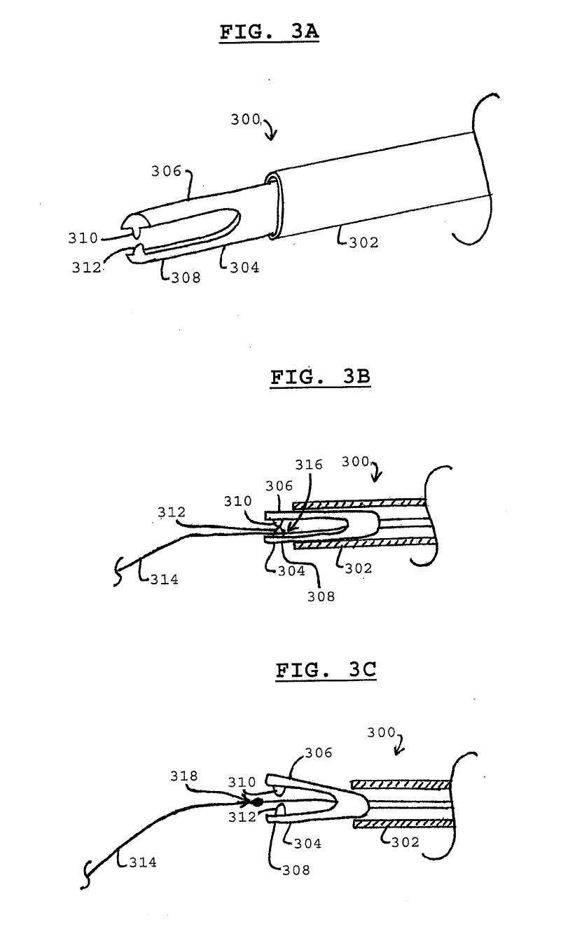

Still further variations of knot pushers may be used. For example, FIGS. 7A and 7B show a knot pusher comprising a swiveling knot-retaining post. Referring first to FIG. 7A, knot pusher (700) comprises an elongated member (702) having a proximal portion (704) and a distal portion (706). In distal portion (706), knot pusher (700) comprises a knot-retaining post (708) positioned in a recess (710) of elongated member (702). A lockout wire (712) is slidably positioned within a lumen (714) of elongated member (702). In FIG. 7A, lockout wire (712) extends throughout lumen (714), and thereby maintains post (708) in an upright position. However, when lockout wire (712) is pulled proximally (in the direction of arrow (A2)), post (708) is allowed to swivel (in the direction of arrow (A3)) from its upright position to a horizontal position. The resulting configuration of knot pusher (700) is shown in FIG. 7B. While a swiveling lockout mechanism is described, other configurations of lockout mechanisms may be used, as appropriate. Moreover, lockout mechanisms may be manually or automatically actuated.

In FIG. 7A, knot pusher (700) is disposed within a delivery sheath (716) that may be used to deliver knot pusher (700) to a target site. However, prior to knot pusher (700) being positioned within the sheath, a knot may be relatively easy tied around post (708). For example, FIG. 7A shows an open knot (718) that has been formed from a tether (720) around post (708). Once knot (718) has been formed, knot pusher (700) may be positioned within delivery sheath (716), and may be delivered to a target site by being advanced over tether (720) (e.g., while tether (720) is tensioned). Of course, in some variations, one or more knots may be formed when knot pusher (700) is at least partially disposed within delivery sheath (716). As knot pusher (700) is advanced to the target site, open knot (718) typically is forced to move along with the knot pusher. Referring now to FIG. 7B, once knot pusher (700) has reached the target site, delivery sheath (716) may be at least partially withdrawn from knot pusher (700). Next, lockout wire (712) may be pulled proximally such that post (708) is allowed to swivel, thereby releasing open knot (718). Tether (720) may then be tightened to form a closed knot (722).

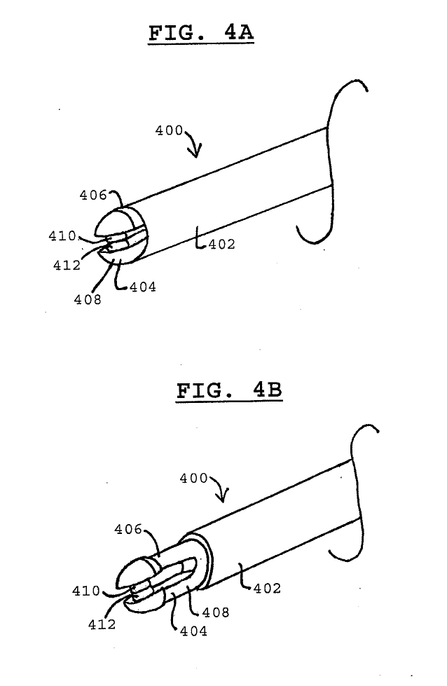

FIGS. 7A and 7B illustrate just one variation of a knot pusher having a movable knot-retaining post. Other appropriate variations may also be used. As an example, FIGS. 8A and 8B show a distal portion of a knot pusher (800) including a body portion (802) and a swiveling member (808). Body portion (802) defines a tether lumen (804) and a groove (812), and swiveling member (808) comprises a post (806) and a rod (810). In FIG. 8A, post (806) is in its upright position, such that it may be used to deliver an open knot to a target site. When post (806) is in its upright position, rod (810) is disposed within groove (812) of body portion (802). In FIG. 8B, however, post (806) has swiveled to its horizontal position, such that rod (810) is no longer disposed within groove (812) of body portion (802). When post (806) is in its horizontal position, the open knot may be released and converted into a closed knot.

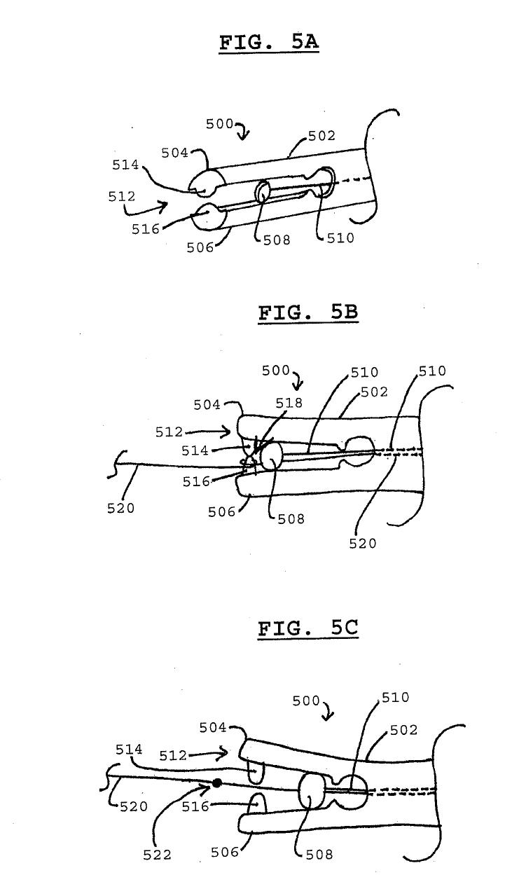

FIGS. 9A and 9B show another variation of a knot pusher. As shown there, a knot pusher (900) comprises a body (902) located at a distal end (904) of an elongated member (906). Body (902) includes a recessed region (908), in which a first roller (910) and a second roller (912) are located. Knot pusher (900) also comprises a swiveling tether guide (914) capable of swiveling toward and away from first and second rollers (910) and (912).

FIG. 9A shows knot pusher (900) in its locked configuration. As shown there, a tether (916) has been wrapped around first and second rollers (910) and (912) to form an open knot (918). Remaining tether material has been routed through a tether aperture (920) in body (902) and pulled proximally (e.g., to eliminate slack tether material). When knot pusher (900) is in its locked configuration, tether guide (914) is positioned between first and second rollers (910) and (912), thereby locking a portion of open knot (918) within recessed region (908). In this locked configuration, knot pusher (900) may be advanced distally over tether (916) to the target site. Open knot (918) is typically forced to move distally, as well, rolling along first and second rollers (910) and (912). In some variations, knot pusher (900) may provide an indication, such as a tactile indication, once the target site has been reached. For example, in a procedure involving tethered anchors, first roller (910) may come into contact with the proximal-most tethered anchor, thereby indicating that the target site has been reached.

To unlock knot pusher (900), tether guide (914) may be swiveled in the direction of arrow (A4), assuming the position shown in FIG. 9B. Tether guide (914) may then be rotated underneath tether (916) in the location of open knot (918), thereby pushing the open knot off of first and second rollers (910) and (912). Open knot (918) may then be formed into a closed knot (e.g., by pulling on the proximal end of tether (916)).

Additional variations of knot pushers or other knot formation and/or advancement devices may be used. As an example, some of the above knot pushers have posts or rollers located in recessed regions. This positioning in recessed regions may help to maintain the overall profile of the knot pusher. However, in certain variations, a knot pusher may comprise one or more posts, rollers, and/or other knot-retaining components that are not disposed in such a recess. As another example, in some variations, a knot pusher may comprise both a clamping member and one or more posts, rollers, and/or other knot-retaining components.

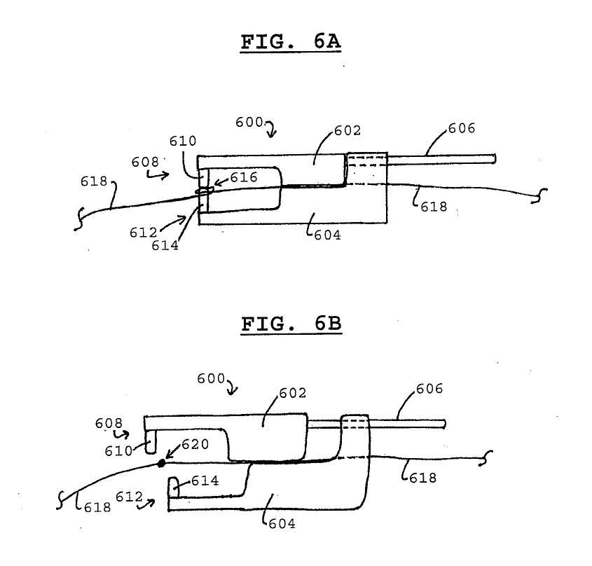

Additionally, any appropriate knot configuration may be used with the devices, methods and kits described here. For example, FIG. 10A depicts a variation of an open knot (1000) formed in a tether (1002). Any suitable variation of a knot pusher or other device may be used to form and/or advance open knot (1000). As an example, FIG. 10B shows a knot pusher (1004) comprising an outer tubular member (1006) and an inner elongated member (1008) slidably disposed within a lumen (not shown) of outer tubular member (1006). As shown in FIG. 10B, a portion (1010) of inner elongated member (1008) extends from the distal end (1012) of outer tubular member (1006), and open knot (1000) is formed around the portion. During use, open knot (1000) may, for example, be advanced to a desired target site. At the target site, portion (1010) of inner elongated member (1008) may be retracted into the lumen of outer tubular member (1006), and/or outer tubular member (1006) may be distally advanced over portion (1010). This retraction and/or advancement may effectively push open knot (1000) off of portion (1010). Tether (1002) may then be tensioned to convert open knot (1000) into a closed knot (not shown), such that the tether becomes locked.

Some variations of knot pushers or other knot formation and/or advancement devices may comprise one or more cone- or funnel-shaped components. Such components may be used, for example, to help seat or otherwise position one or more knots while the knot(s) are advanced to a target site. They may also provide for relatively easy and streamlined advancement of the knot pushers or other devices to a target site. Other appropriate shapes aside from cones and funnels may also be used.

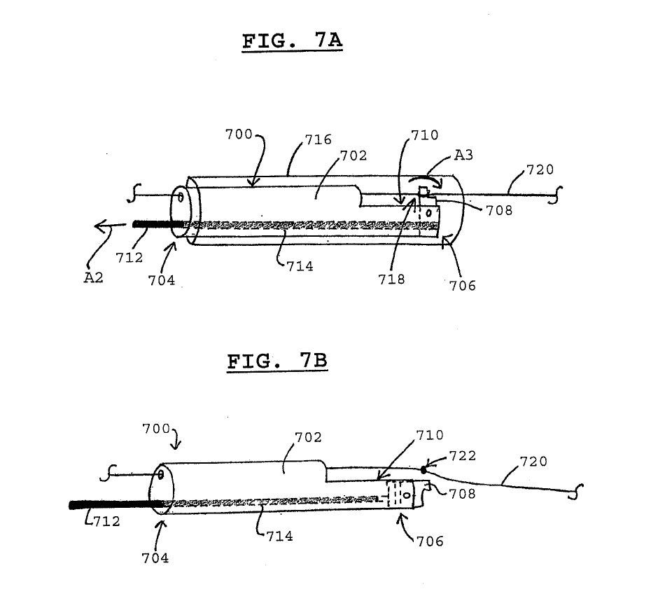

FIG. 11A shows an exemplary cone-shaped component (1100), and FIG. 11B depicts a device (1102) comprising the cone-shaped component coupled to a distal end (1106) of an elongated member (1104). While cone-shaped component (1100) is coupled to elongated member (1104), in some variations a component may be integral with an elongated member. For example, the component and the elongated member may be integrally molded.

FIG. 11C provides an illustrative view of cone-shaped component (1100) in use. As shown there, a knot (1108) formed of two tethers (1110) and (1112) may be pushed in the direction of arrow (1114) (e.g., using a finger or a pushing member) such that the knot is seated in component (1100). Component (1100) may provide interference with the force or movement used to seat knot (1108), and may thereby help in the formation of a relatively tight and strong seated knot. In some variations, component (1100) may comprise one or more apertures therethrough to enhance the positioning of knot (1100). As an example, component (1100) may comprise a closed distal end having two apertures therethrough, such that each of the tethers may be threaded through an aperture. In certain variations, component (1100) may be releasably coupled to elongated member (1104), such that the component and one or more knots seated in the component may be released and left behind at a target site. While knot (1108) is depicted as positioned within component (1100) during use, in some variations, a knot and/or one or more tether strands may alternatively or additionally be positioned around the component, or in another location relative to the component.

A cone-shaped component such as component (1100) may have any appropriate size. In some variations, the dimensions of such a component may be selected based on the size of the knot or knots to be advanced and/or formed, and/or on the characteristics of the target site. In certain variations, a wall portion (1116) (FIG. 11C) of the component may be relatively thin (e.g., from about 0.01 inch to about 0.04 inch). This may, for example, result in a relatively low overall profile and size for the component, as well as for a device comprising the component.

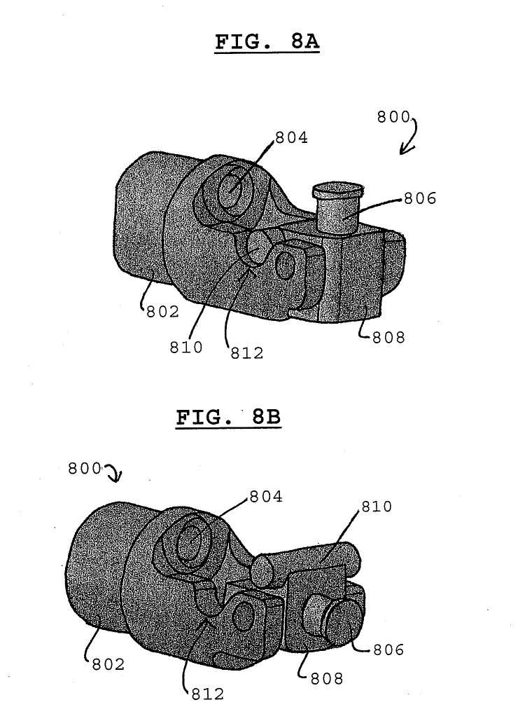

Certain cone-shaped configurations have been depicted; however, other cone-shaped configurations may also be employed. As an example, in some variations, a half-cone may be used. For example, FIG. 12 shows a device (1200) comprising a half-cone component (1202) coupled to a semitubular elongated member (1204). Device (1200) may, for example, allow for seating of a slip-knot, such that the slip-knot may be advanced to a target site. In certain variations, device (1200) may further comprise one or more features allowing for tracking of a knot (e.g., an open knot) within semitubular elongated member (1204). In some variations, device (1200) may function as a shield that may be used in conjunction with a knot pusher or other knot formation and/or advancement device. The shield may, for example, protect the knot pusher or other device from interference with leaflets and/or other tissue, and may thereby decrease the likelihood of the knot pusher or other device becoming stuck during use. It should be understood that any of the devices and/or components described here may function as a shield in this way, as appropriate.

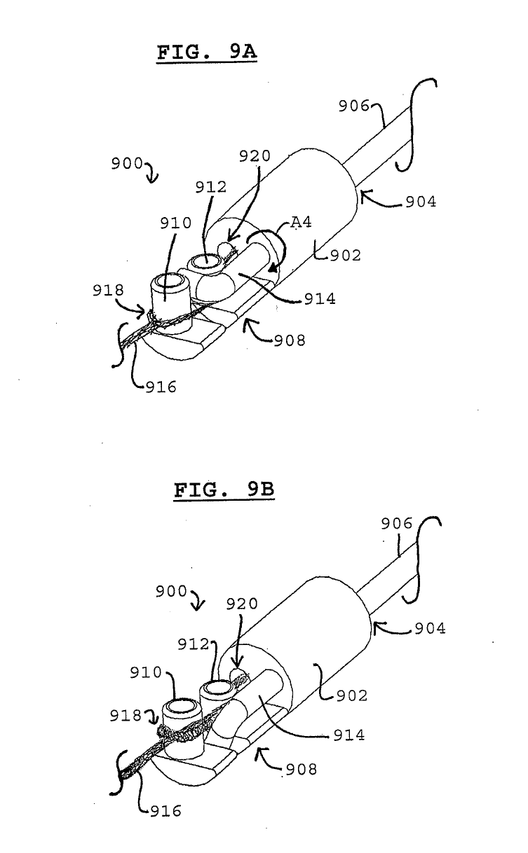

Cone-shaped components and other components may comprise any appropriate features, such as rounded surfaces, edges, etc., and generally may have any shape that is suitable for being advanced to the applicable target site. In some variations, such components may have one or more grooves, slots, apertures, protrusions, and/or other features that may help in the positioning and/or routing of a tether and/or knot. For example, FIG. 13 shows a component (1300) in the shape of an edged cone. As shown there, component (1300) comprises a hollow region (1302) and wall portions (1304) and (1306) each having a slot (1308) and (1310) therein, respectively. During use, a knot may, for example, be positioned within hollow region (1302), and tether strands from the knot may be routed through slot (1308) and/or slot (1310). In certain variations, the tether strands may form a friction fit with the slots, such that the tether strands are relatively securely positioned during use. Once the knot has been properly seated, component (1300) may be advanced (e.g., along a tether in which the knot is formed) to a target site. By routing the tether strands through the slots and thereby spreading the tether strands out, a relatively tight and secure knot may be formed near the base of the hollow cone. In some variations, component (1300) may be releasably coupled to one or more other components of a knot formation and/or advancement device, such that the component may be released from the rest of the device and left behind at the target site. While component (1300) is depicted as having two slots (1308) and (1310), other variations of components may have just one slot, may have more than two slots, or may not have any slots at all. Moreover, some variations of components may comprise at least one slot in combination with one or more other features.

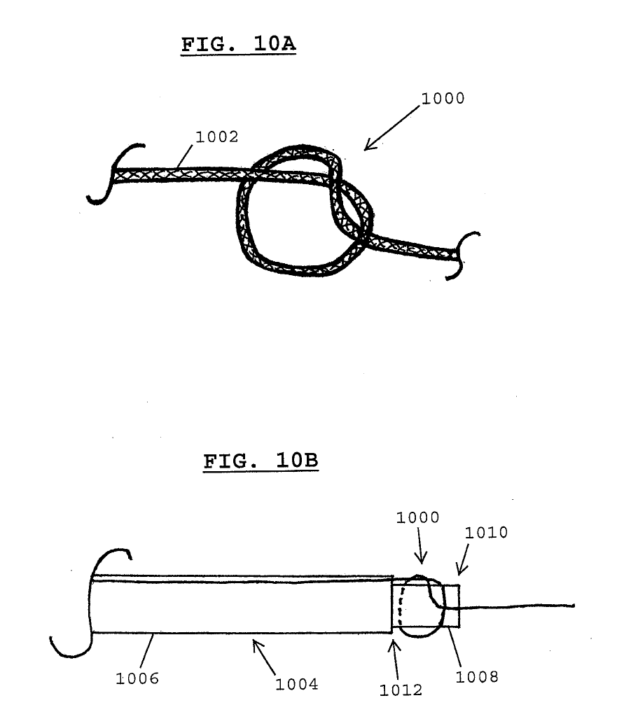

In certain variations, a knot pusher or other knot formation and/or advancement device may comprise one or more collapsible components. Such components may enhance the flexibility and/or maneuverability of the device, and/or may enhance the ability of the device to hold a knot. FIG. 14A shows a cross-sectional view of an exemplary cone-shaped component (1400) comprising a hollow-region (1402) and a wall portion (1404) having two apertures (1406) and (1408) therethrough. Component (1400) also comprises a wall portion (1410). As shown in FIG. 14A, a knot (1412) formed between two tethers (1414) and (1416) is positioned within hollow region (1402). Additionally, tether (1414) is routed through aperture (1406), and tether (1416) is routed through aperture (1408). Referring also now to FIG. 14B, during use, a force may be applied to knot (1412) in the direction of arrow (1418) (e.g., by pushing against the knot with a finger). As a result, wall portion (1410) may collapse (FIG. 14B), thereby seating knot (1412). This collapsing of wall portion (1410) may allow the strands to spread out more and/or may facilitate pushing of the knot to the base of the cone. Once the knot has been seated, component (1400) may, for example, be advanced to a target site.

Cone-shaped components or other components may be designed to be left behind at a target site, as discussed above, or may be designed for removal from a target site. For example, FIG. 14C provides a cross-sectional view of a relatively wide cone-shaped component (1450) comprising a wall portion (1452) that is temporarily held together by a suture (1454). As shown, suture (1454) is effectively sewn between different regions of wall portion (1452), thereby holding the different regions together. For example, the different regions of the wall portion may each have an edge, and the edges may be stitched to each other using the suture. In some variations, the cone-shaped component may have a slit in the wall portion that at least partially separates the different regions from each other, and the slit may be temporarily closed by being stitched together with the suture. A knot (1456) in suture (1454) prevents suture (1454) from slipping out, and thereby prevents the different regions of wall portion (1452) from prematurely separating. While a single knot is shown here, other variations of components may comprise multiple knots, and/or may comprise one or more other features that are intended to prevent the components from prematurely separating. If so desired (e.g., upon reaching a target site), suture (1454) may, for example, be cut to allow the different regions of wall portion (1452) to separate from each other. Cone-shaped component (1450) may then be withdrawn, leaving knot (1412) behind.

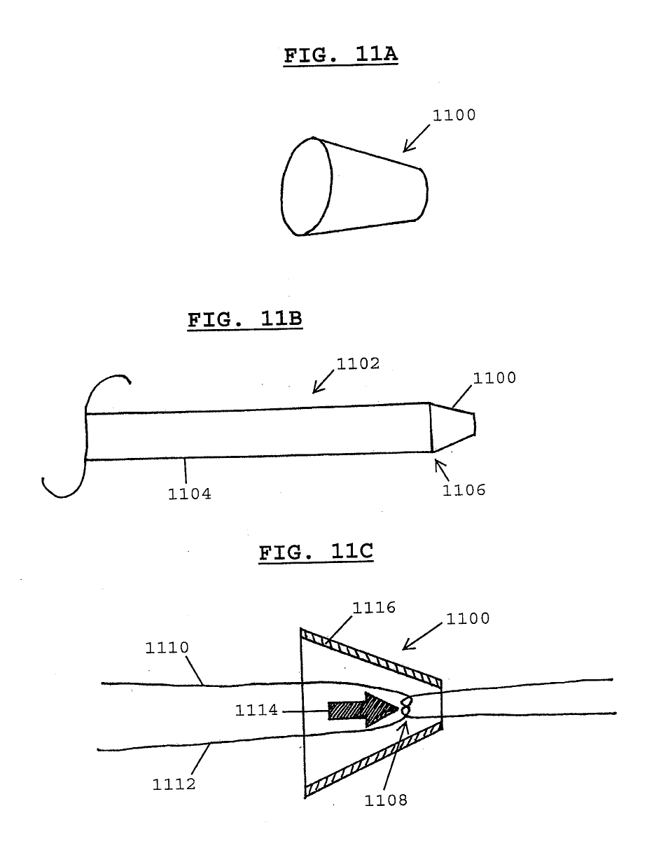

Additional variations of knot formation and/or advancement devices and components may be used. For example, FIG. 15A shows a shielded knot pusher (1500) comprising a half-cone (1502) coupled to an elongated member (1504). Elongated member (1504) is off-center with respect to half-cone (1502); however, other variations of devices or components may comprise a half-cone and an elongated member that is centered with respect to the half-cone. In use, a knot (not shown) may, for example, be positioned within and/or around half-cone (1502) and/or around elongated member (1504), and shielded knot pusher (1500) may be used to advance the knot to a target site. In cases in which the knot is positioned within half-cone (1502), the half-cone may function as a shield, and may thereby protect the knot. While a half-cone is depicted, other variations of components having different configurations may also be used. As an example, FIG. 15B depicts a shielding component (1550) having a relatively rounded hood shape, which may be atraumatic and/or relatively easy to advance through a tortuous pathway. In certain variations, a shield may be expandable; for example, a shield may be in the form of an expandable cone.

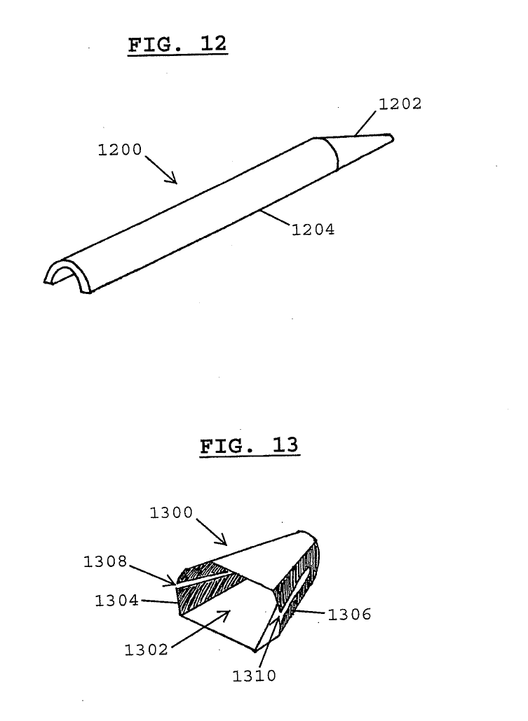

In some variations, a knot pusher or other knot formation and/or advancement device may comprise one or more funnel-shaped components. Such components may, for example, provide a relatively good shielding effect (e.g., thereby protecting the knot(s) from the surrounding tissue, and/or the surrounding tissue from the knot(s)). As an example, FIG. 16A shows a device (1600) for the formation and/or advancement of one or more knots, where the device comprises an elongated knot pusher (1602) and a funnel-shaped shield (1604) positioned around a distal portion (1606) of the elongated knot pusher. The funnel-shaped shield may, for example, provide protection to elongated knot pusher (1602) and/or to one or more knots being carried or otherwise supported by the knot pusher. Shield (1604) is depicted as being used with knot pusher (1602); however, shield (1604) may alternatively be used with other suitable variations of knot pushers or knot formation and/or advancement devices.

While shield (1604) has a particular funnel shape, other funnel shapes may also be used. As an example, FIG. 16B shows a device (1650) for the formation and/or advancement of one or more knots, where the device comprises an elongated knot pusher (1652) and a shield (1654) that is shaped somewhat like a half-funnel. Shield (1654) may, for example, help to provide protection to at least a portion of elongated knot pusher (1652) (e.g., shielding it from surrounding tissue during advancement), to one or more knots formed on and/or carried by elongated knot pusher (1652), and/or to surrounding tissue (e.g., by presenting a relatively atraumatic surface during advancement of elongated knot pusher (1652)). Other variations of shields and other types of protection mechanisms may provide similar advantages. Devices (1600) and/or (1650), as well as other devices described herein, may in some variations provide for one-handed operation, and may therefore be relatively easy to use. While certain configurations of shields have been depicted, other configurations of shields may, of course, alternatively or additionally be used.

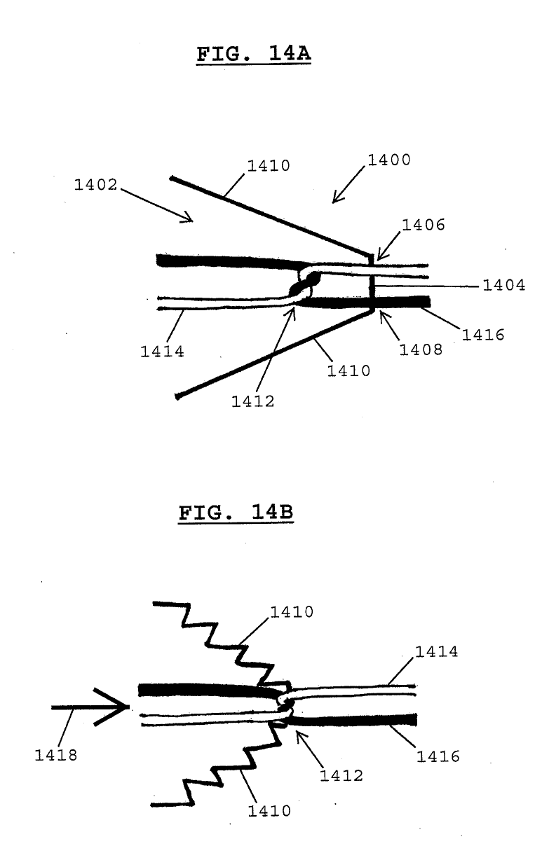

FIG. 17 shows another variation of a knot pusher (1700), comprising an elongated member (1702) having a lumen (not shown) therethrough, such that the elongated member is capable of being advanced over a tether, for example. Elongated member (1702) comprises a distal portion (1704) having a groove (1706) therein with an opening (not shown) to the lumen. During use, and as shown, a tether (1710) may be passed through the opening and into the lumen. The tether may comprise one or more knots therein having a size that prevents the knots from fitting within the lumen. In some variations, the knot(s) may be tied around a post or other feature (not shown) near the opening. The knot(s) may be capable of moving around the post or other feature. In use, knot pusher (1700) may then be advanced over tether (1710) and toward a target site, thereby pushing the knot(s) toward the target site, as well.

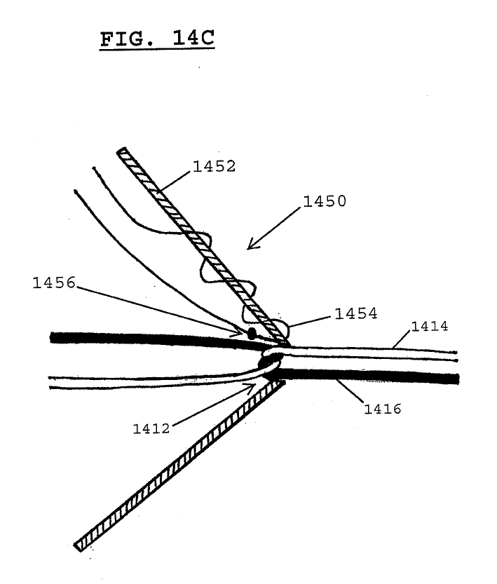

A further variation of a knot pusher is depicted in FIG. 18. As shown there, a knot pusher (1800) comprises an elongated member (1802) having a rounded distal portion (1804), and a post (1806) that extends from a surface (1808) of the elongated member. In use, and as shown, a knot (1810) formed of one or more tethers (here, tethers (1812) and (1814)) may be positioned against or around post (1806), and elongated member (1802) may be pushed distally toward a target site, thereby advancing knot (1810) to the target site. Once at the target site, elongated member (1802) may be withdrawn, and tethers (1812) and (1814) may be tensioned to tighten knot (1810). In certain variations, knot pusher (1800) may further comprise a sliding or retractable hood or shield (not shown) that may be temporarily positioned over post (1806), and may thereby shield or otherwise protect knot (1810) as it is being advanced to a target site. Once at the target site, the hood or shield may be retracted or otherwise removed to expose the knot, so that the knot may be left at the target site.

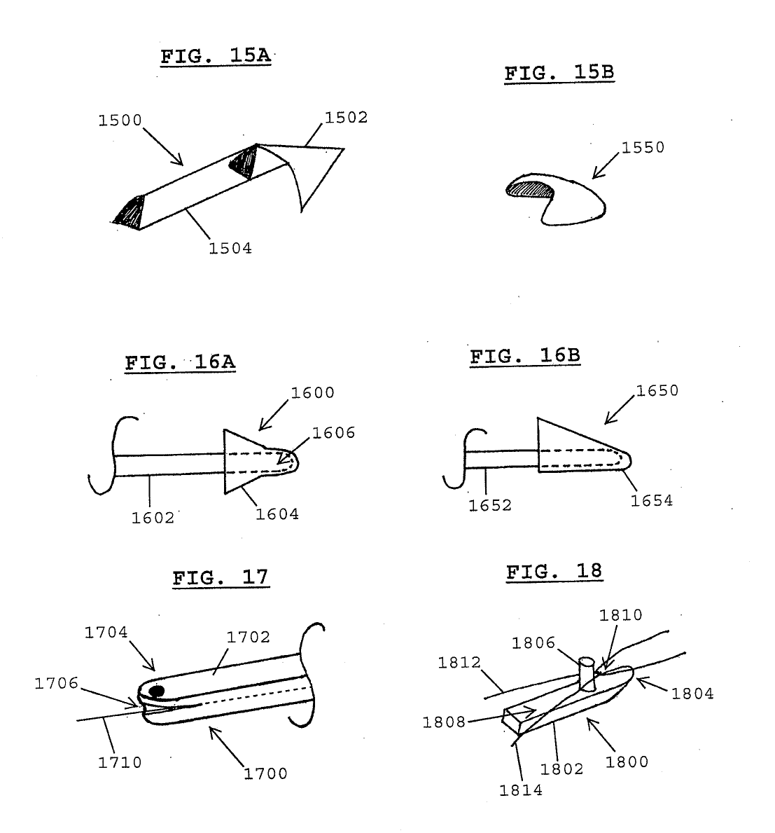

FIG. 19A shows a top view of another variation of a knot pusher (1900). As shown there, knot pusher (1900) comprises a bullet-shaped distal portion (1902) having an elongated groove (1904) and a knot-positioning aperture (1906). FIG. 19B provides a side view of knot pusher (1900), with elongated groove (1904) and aperture (1906) depicted in phantom. In use, and as shown in FIG. 19C, a knot (1908) (e.g., a half-knot or a slip knot) in a tether (1910) may be positioned within aperture (1906), and the surrounding tether portions may be positioned within groove (1904). Knot pusher (1900) may then be advanced along tether (1910), such that knot (1908) is advanced along with it. In some variations, by positioning knot (1908) within aperture (1906), knot pusher (1900) may help to keep the knot down, and may thereby prevent the knot from catching tissue, such as heart valve leaflets.

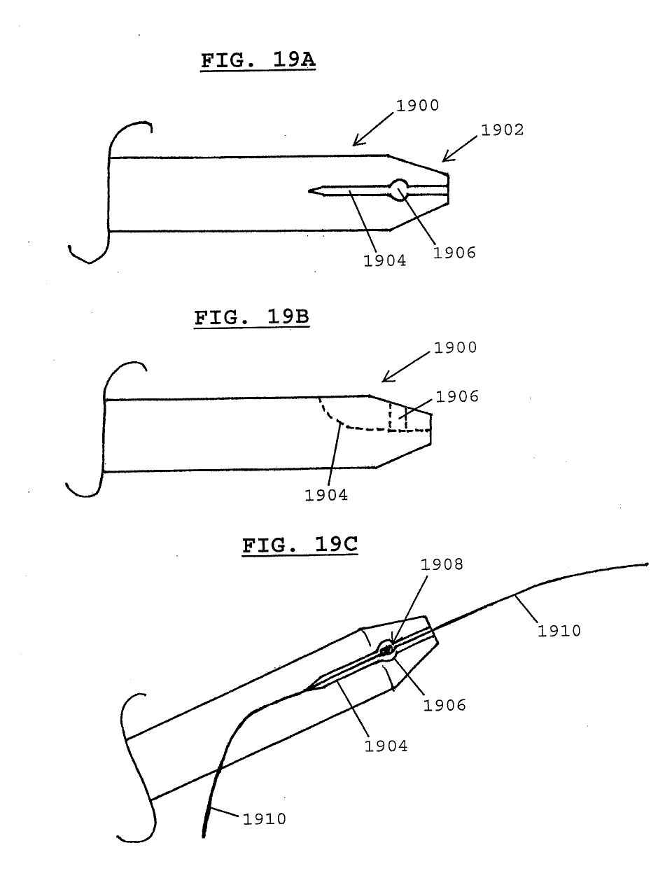

FIG. 20A shows another variation of a device (2000) which may be used to form one or more knots and/or to advance one or more knots to a target site. As shown there, device (2000) comprises an elongated knot pusher (2002) and a corresponding shield (2004) that fits over at least a portion of the knot pusher. FIG. 20B provides a top view of shield (2004), which comprises a hooded distal portion (2006) configured to protect the distal portion (2008) of knot pusher (2002) during use. Shield (2004) comprises slots (2010) and (2012), which are designed to allow distal portion (2006) to expand as shield (2004) is retracted over knot pusher (2002), and/or as knot pusher (2002) is pushed out of shield (2004). For example, shield (2004) may be used to protect knot pusher (2002) and the knot(s) it is carrying during advancement to a target site, and may then be retracted upon reaching the target site, to allow the knot(s) to be released at the target site. While shield (2004) is depicted as having two slots (2010) and (2012), any appropriate number of slots and/or other features that permit for such expansion may be used.

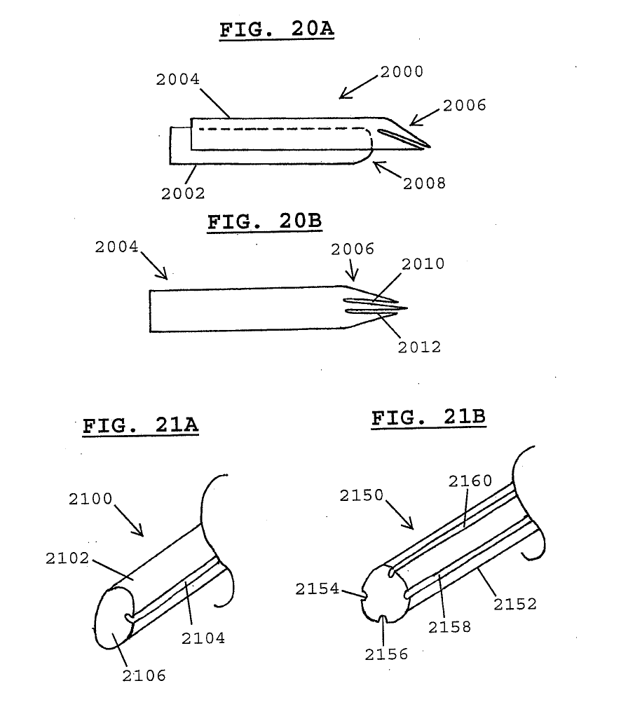

In certain variations, a knot pusher may comprise one or more grooves and/or other features that may be used, for example, to route portions of the tether (or another element in which the knot is formed). For example, FIG. 21A shows a knot pusher (2100) comprising an elongated member (2102) having a groove (2104) therein. While groove (2104) is depicted as extending into the distal end (2106) of elongated member (2102), in some cases such a groove may not extend into the distal end of the elongated member. This may, for example, allow a knot that is positioned around the distal end to be positioned flush to the tissue as the knot is being advanced (e.g., thereby maintaining intended fixation of the tissue). While not shown in FIG. 21A, in some variations, knot pusher (2100) may further comprise one or more additional components, such as a shield over at least a portion of elongated member (2102). Additionally, a knot pusher may comprise more than one groove. As an example, FIG. 21B shows a knot pusher (2150) comprising an elongated member (2152) having four grooves (2154), (2156), (2158) and (2160) therein. The grooves are shown as being approximately evenly radially spaced around elongated member (2102); however, other variations of devices may comprise grooves or other features that are not evenly spaced. The number of grooves or other features in a device, as well as their positioning, may be selected based, for example, on the number of tether strands involved, the type of knot involved, etc.

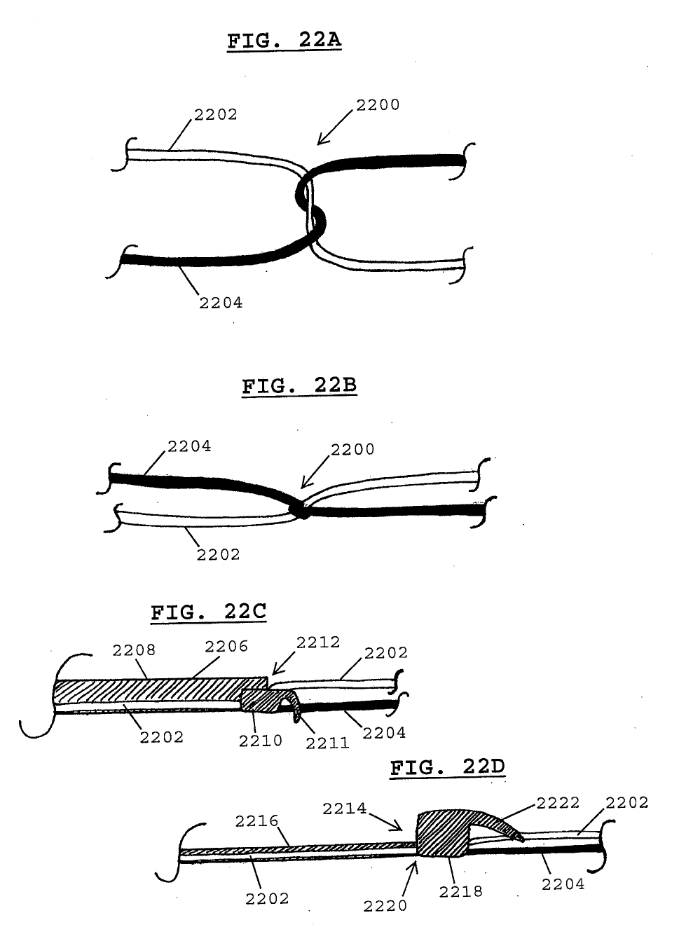

FIGS. 22A-22D depict variations of knot configurations and knot pushers that may be used with the knot configurations. Alternatively, the knot configurations may be used with other knot pushers or devices, and the knot pushers may be used with other knot configurations. First, FIGS. 22A and 22B show different views of a knot (2200) formed from two tethers (2202) and (2204). In FIG. 22C, knot (2200) has been positioned on a knot pusher (2206) comprising an elongated member (2208) and a shield member (2210) coupled to a distal portion (2212) of elongated member (2208). Shield member (2210) comprises a curved portion (2211) that may be used, for example, to keep heart valve leaflets and/or other tissue out of the way of knot (2200) as the knot is being advanced to a target site. FIG. 22D similarly shows knot (2200) positioned on another knot pusher (2214), which comprises an elongated member (2216) and a shield member (2218) coupled to a distal portion (2220) of elongated member (2216). As shown there, shield member (2218) comprises a curved portion (2222) that may function to protect knot (2200), as described above. While shield members (2210) and (2218) each are depicted as comprising one curved portion, other variations of shield members may comprise more than one curved portion, and/or may be differently configured in one or more other ways.

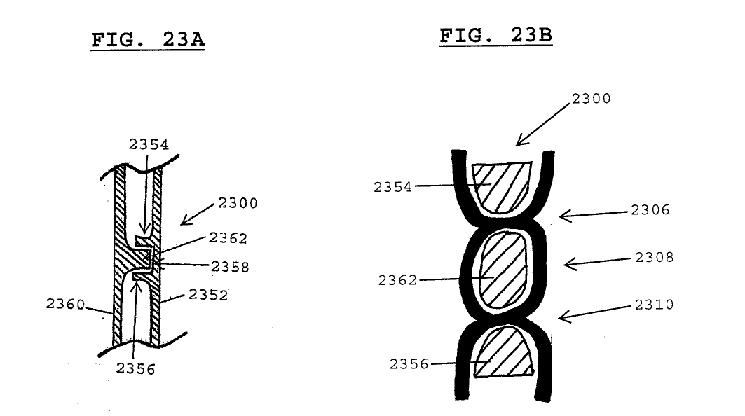

In some variations, a knot pusher may have a splitting configuration that may be used to advance and/or form one or more knots. For example, in some cases, the knot pusher may be used to advance a half-knot to a target site, and/or to form a square knot from a half-knot. FIG. 23A provides a side cross-sectional view of one variation of such a knot pusher (2300), which comprises a first portion (2352) comprising two projections (2354) and (2356) with a groove (2358) therebetween, as well as a second portion (2360) comprising a projection (2362) configured to fit within the groove.

When knot pusher (2300) is in use, a knot may, for example, be woven around projections (2354) and/or (2356), and projection (2362) may temporarily engage with groove (2358), to hold the knot in place (e.g., during advancement to a target site). As an example, FIG. 23B provides a top cross-sectional view of knot pusher (2300), showing the positioning of projections (2354), (2362) and (2356) when projection (2362) is engaged with groove (2358). As depicted in FIG. 23B, a half-knot (2308) is positioned around projections (2354), (2362) and (2356). As half-knot (2308) is advanced to a target site, the projections may maintain the half-knot in an untightened configuration and may be used to essentially roll the half-knot down to the target site. Once at the target site, projection (2362) may be disengaged from groove (2358), and the half-knot may be allowed to tighten and close (e.g., the top portion (2306) of the half-knot may tighten around the bottom portion (2310) of the half-knot, thereby forming a square knot). Of course, other suitable knot configurations may also be used with knot pusher (2300). Additionally, while knot pusher (2300) comprises three projections and one groove, any appropriate number or configuration of projections and/or grooves may be used in a device, as appropriate.

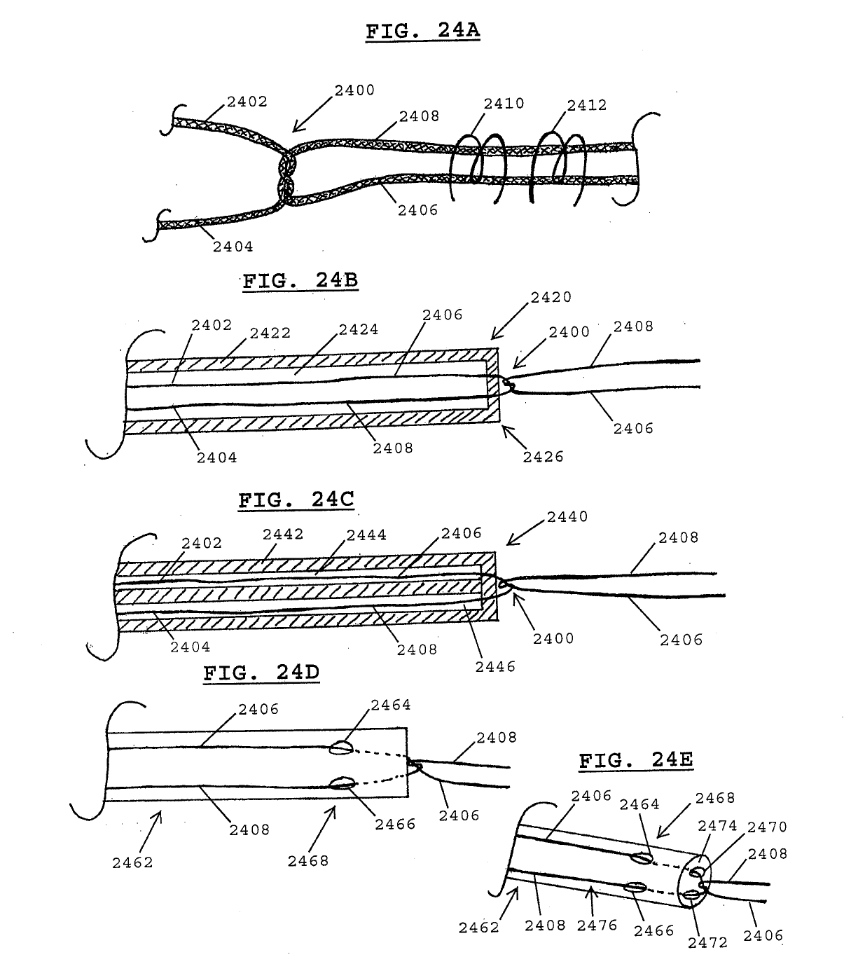

Additional variations of half-knot pushers are depicted in FIGS. 24B-24E. Referring first to FIG. 24A, a half-knot (2400) is formed from two tethers (2402) and (2404), where two strands (2406) and (2408) of the half-knot are engaged with two tissue anchors (2410) and (2412). Any appropriate variation of a knot pusher may be used to advance half-knot (2400) or other suitable knots to a target site, such as a specific anchor. Once the half-knot has reached the target site, it may be tensioned to form a closed knot, to maintain tension in at least one of the tethers, and/or to help maintain the position of one or more anchors.

FIG. 24B shows a knot pusher (2420) comprising an elongated tubular member (2422) having a lumen (2424) therethrough. As shown, knot pusher (2420) is being used to advance half-knot (2400). Strands (2406) and (2408) of tethers (2402) and (2404) have been positioned within lumen (2424) and threaded through a distal end (2426) of elongated tubular member (2422). Half-knot (2400) is positioned adjacent to distal end (2426). In some cases, strands (2406) and (2408) may be threaded through apertures (not shown) in distal end (2426), and/or may be otherwise stabilized (e.g., by being positioned in one or more grooves in an internal wall of the elongated tubular member). Once half-knot (2400) has been properly positioned with respect to knot pusher (2420), the knot pusher may be used, for example, to advance the half-knot to a target site.

Of course, knot pushers having other configurations may be used. For example, while knot pusher (2420) comprises a single lumen (2424), some variations of knot pushers may comprise more than one lumen (or may not comprise any lumens at all). As an example, FIG. 24C shows a dual-lumen knot pusher (2440) comprising an elongated member (2442) comprising a first lumen (2444) and a second lumen (2446). As shown in FIG. 24C, strands (2406) and (2408) of tethers (2402) and (2404) have each been positioned within a lumen—i.e., strand (2406) has been positioned within first lumen (2444), and strand (2408) has been positioned within second lumen (2446). Positioning the strands within different lumens in this way may, for example, enhance the organization and efficiency of the knot pushing (e.g., limiting the likelihood of strands becoming tangled during use).

In certain variations, one or more tether strands may be routed through the length of a knot pusher (e.g., within one or more lumens of the knot pusher). Alternatively or additionally, in some variations, one or more tether strands may be routed through only a portion of a knot pusher. For example, a tether strand may be routed into and out of a lumen of a knot pusher, in some cases multiple times. FIGS. 24D and 24E depict an exemplary tether-routing configuration. As shown there, tether strands (2406) and (2408) have been routed through two apertures (2470) and (2472) in a wall portion (2474) of a knot pusher (2462), through a portion of a lumen (not shown) of the knot pusher, out through two apertures (2464) and (2466) in a distal portion (2468) of the knot pusher, and along the exterior surface (2476) of the knot pusher. By being routed through multiple apertures and into and out of knot pusher (2462) in this way, strands (2406) and (2408) may be relatively securely positioned with respect to the knot pusher. This may, for example, allow for highly controlled and steady knot advancement.

As described briefly above, in some methods, it may be necessary to load a tether into a device, such as a knot pusher. Various methods and/or devices may be used to accomplish this loading. As an example, in certain variations, a tether may be loaded into a device using a lasso comprising a loop. One end of the tether may be threaded through the loop of the lasso. The lasso may then be pulled along a longitudinal axis of the device to load the tether into the device. Alternatively, the lasso may be pulled through a side hole in a device to load the tether into the device. In certain variations, a lasso may be used to help tie a knot, such as an open knot, in a tether (e.g., where the knot is tied around a portion of a knot pusher or other device). Lassos may be made from, for example, conventional materials such as wire, suture, cable, string, or a monofilament. A lasso may comprise a loop (as described above), a hook, a coil, a tube, an elongate element with a hole, or any other structure or material that can “grab” a tether. Tether-loading devices are described, for example, in U.S. patent application Ser. Nos. 11/232,190 (published as US 2006/0190030 A1), 11/270,034 (published as US 2006/0122633 A1) and 12/577,044 (filed on Oct. 9, 2009), all of which are hereby incorporated by reference in their entirety.

While methods of using one locking device in a tether-locking procedure have been described, it should be understood that some variations of methods may include using multiple locking devices to lock one or more tethers. The locking devices may be the same as each other, or different from each other. As an example, two or more different knot pushers may be used to lock one or more tethers. As another example, a knot pusher and another different type of locking device may be used to lock one or more tethers. For example, a locking device comprising a plug configured to fit within a tubular member may be used in conjunction with a knot pusher to lock a tether. Locking devices are described, for example, in U.S. patent application Ser. No. 12/480,568 (published as US 2010/0049213 A1), and in U.S. patent application Ser. No. 12/577,044, filed on Oct. 9, 2009, both of which are hereby incorporated by reference in their entirety.

In some cases, a knot pusher or other knot advancement and/or formation device may be used in conjunction with one or more devices that help to position, realign, or otherwise manipulate tissue. This may, for example, enhance the efficiency and ease of use of the knot pusher or other device. As an example, tissue may be manipulated to enhance the accessibility of a target site prior to advancement of a knot pusher to the target site.

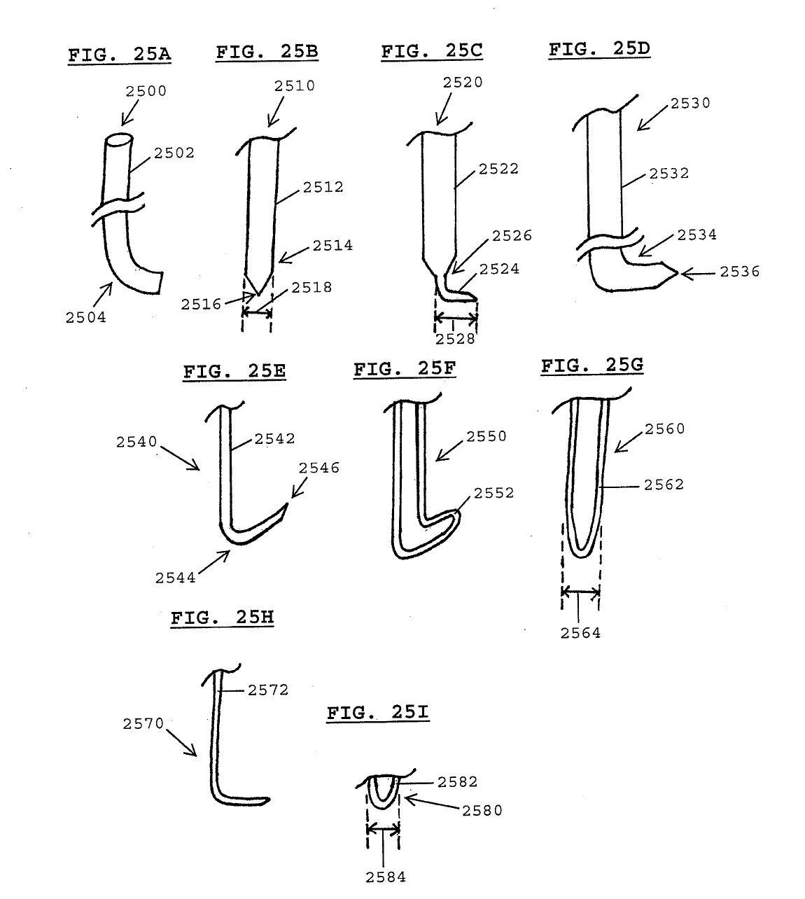

For example, in certain variations, a retractor may be used in conjunction with a knot pusher or other device. The retractor may, for example, be used to lift a valve leaflet to expose a target area for knot tying. FIG. 25A depicts a side view of one variation of an exemplary retractor (2500) comprising an elongated member (2502) having a curved distal portion (2504). Retractor (2500) or other retractors of tissue manipulation devices may comprise any appropriate material or materials, such as one or more metals, metal alloys (e.g., stainless steel), and/or polymers or plastics (e.g., acrylic). For example, in some variations, retractor (2500) may be made from a stainless steel tube (e.g., having a diameter of 0.185 inch). FIG. 25B shows a front view of another variation of a retractor (2510), which comprises an elongated member (2512) having a distal portion (2514) terminating in a pointed distal end (2516). As shown there, distal portion (2514) has a width (2518) which may be, for example, about 0.285 inch. The dimensions of various components or portions of a retractor may depend, for example, on the characteristics of the relevant target anatomy. As an example, a retractor may be dimensioned to navigate between chordae tendineae relatively easily.

Some variations of retractors may comprise one or more hook-like components or other components comprising one or more projections or protrusions, which may be used to engage tissue. For example, FIG. 25C provides a side view of a variation of a retractor (2520) comprising an elongated member (2522) and a hooked projection (2524) extending from a distal end (2526) of the elongated member. As shown, hook projection (2524) has a length (2528) which may be, for example, 0.36 inch.

FIG. 25D depicts an additional variation of a retractor (2530) comprising an elongated member (2532) having a curved distal portion (2534) with a pointed distal end (2536). In some variations, retractor (2530) may be made from an acrylic rod (e.g., having a diameter of 0.128 inch).

Of course, retractors may have any appropriate configuration. For example, while FIG. 25E shows a retractor (2540) comprising an elongated member (2542) and a curved distal portion (2544) having a pointed distal end (2546), the curved distal portion points upward, such that it forms more of a hook-like shape than the curved distal portions of previously depicted retractors. The hook-like shape may, for example, be especially well-suited to lifting valve leaflets relatively easily. In certain variations, retractor (2540) may be made from an acrylic rod (e.g., having a diameter of 0.06 inch).

In certain variations, one or more wires or other elongated members may be shaped to form a retractor. As an example, FIG. 25F shows a retractor (2550) made from a wire (2552), and FIG. 25G shows a retractor (2560) made from a wire (2562). As shown in FIG. 25G, retractor (2560) has a width (2564) which may be, for example, about 0.1 inch. In some variations, a wire that is used to form a retractor may have a diameter of 0.029 inch. FIG. 25H shows another variation of a retractor (2570) made from a wire (2572), and FIG. 25I depicts a further variation of a retractor (2580) made from a wire (2582). As shown, retractor (2580) has a width (2584) which may be, for example, 0.094 inch. Wire (2572) and/or wire (2582) may, for example, have a diameter of 0.22 inch. Of course, while exemplary wire and retractor dimensions have been provided, it should be understood that any appropriate dimensions may be used.

Other configurations of retractors or other tissue manipulation devices may be used in a procedure. For example, in some variations, a retractor may comprise multiple projections and/or protrusions. As an example, a retractor may comprise two hooks that may be used to lift two valve leaflets prior to and/or during a procedure (e.g., two different leaflets on either side of a commissure). As another example, in certain variations, a retractor may comprise one or more portions that are strengthened (e.g., by adding to their thickness and/or by using one or more stronger materials in the portion(s)), without adding undesirable bulk to other portions of the retractor.

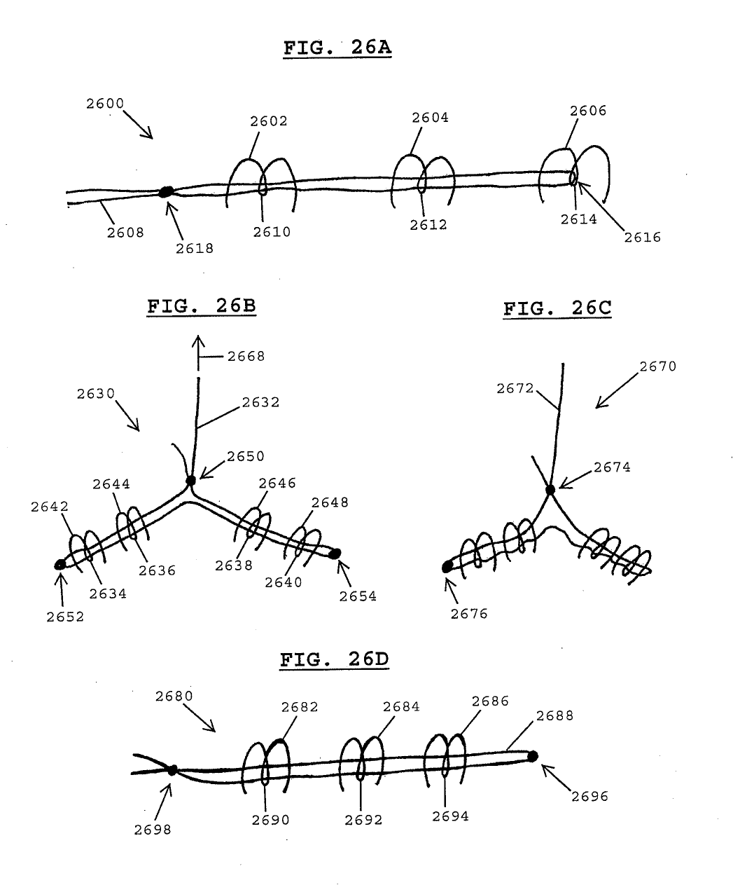

As shown above, in some variations one or more knots may be formed and/or advanced in a tether of a tether-anchor assembly. Tether-anchor assemblies having any appropriate configuration may be used. For example, FIG. 26A shows a tether-anchor assembly (2600) comprising three anchors (2602), (2604) and (2606) and a tether (2608) routed through eyelets (2610), (2612) and (2614) of the three anchors. Of the three anchors, anchor (2602) is the most proximal and anchor (2606) is the most distal. As shown, tether (2608) forms a loop (2616) through eyelet (2614) of anchor (2606), and is tied into a knot (2618) proximal to anchor (2602). During use, tether (2608) may be pulled upon proximally to cinch anchors (2602), (2604) and (2606), thereby bringing the anchors closer together and compressing the tissue therebetween.

FIG. 26B shows another variation of a tether-anchor assembly (2630). As shown there, tether-anchor assembly (2630) comprises a tether (2632) routed through eyelets (2634), (2636), (2638) and (2640) of anchors (2642), (2644), (2646) and (2648) in an inverted “Y” configuration. As shown in FIG. 26B, tether (2632) has three knots (2650), (2652) and (2654) formed therein. Knots (2652) and (2654) may, for example, be fixed knots that may help to prevent tether (2632) from sliding through eyelets (2634) and (2640). Knot (2650) may, for example, be a sliding or adjustable knot that may be used to help cinch anchors (2642), (2644), (2646) and/or (2648) when pulling upon tether (2632) in the direction of arrow (2668). Alternatively, any combination of sliding or adjustable knots and/or fixed knots may be used. For example, in some variations, all of the knots in a tether-anchor assembly may be sliding or adjustable knots.

A tether-anchor assembly may have any suitable number, size and shape of knots, loops and other features. For example, FIG. 26C shows a tether-anchor assembly (2670) that is similar to tether-anchor assembly (2630) of FIG. 26B, but that comprises a tether (2672) having only two knots (2674) and (2676) formed therein. Additionally, tether-anchor assembly (2670) is depicted as having some slack along its length. FIG. 26D shows a tether-anchor-assembly (2680) comprising a proximal anchor (2682), an intermediate anchor (2684) and a distal anchor (2686), as well as a tether (2688) routed through eyelets (2690), (2692) and (2694) of the anchors. Tether (2688) includes a knot (2696) distal of distal anchor (2686), as well as a knot (2698) proximal to proximal anchor (2682). While different variations of tether-anchor assemblies have been depicted in FIGS. 26A-26D, any other suitable tether-anchor assembly may be used in a particular application, as appropriate. In some variations, at least one (e.g., all) of the knots in a tether-anchor assembly may be capable of being adjusted (e.g., to alter the overall shape of the tether-anchor assembly).

As described above, in operation, a knot pusher or other knot formation and/or advancement device may be used to position one or more knots to secure a tether and thereby fix the length of the tether and/or prevent the tether from moving. After the tether has been secured, any excess length of the tether may be cut and removed. Generally, the tether may be cut proximal to the knot or knots. In some cases, it may be desirable to cut the tether as closely as possible to the knot or knots.

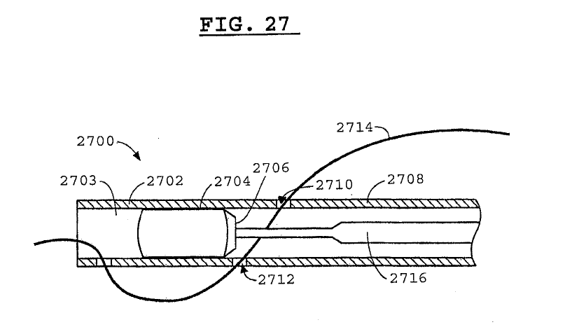

FIG. 27 shows a variation of a device that may be used to cut a tether. As shown there, cutting device (2700) comprises a tubular elongated member (2702) having a lumen (2703), and a cutter (2704) disposed within the lumen of the elongated member. Cutter (2704) has a cutting blade (2706) that faces in a proximal direction. Cutter (2704) may, for example, be in the form of a metal tube having a sharpened edge. Elongated member (2702) comprises a side wall (2708) having two apertures (2710) and (2712). A tether (2714) has been threaded through apertures (2710) and (2712), such that the tether crosses the lumen of the elongated member.

While two side wall apertures are shown, other variations of devices may include a different number of side wall apertures, such as three or four side wall apertures. When it is desired to sever tether (2714), cutter (2704) may be pulled proximally using a pulling member (2716) that is attached to cutter (2704). This causes cutting blade (2706) to contact and sever tether (2714). While cutter (2704) is pulled proximally using pulling member (2716), in some variations, a cutter disposed within the lumen of an elongated member may alternatively or additionally be pushed in a proximal direction. For example, a pushing member may be placed into the elongated member at its distal end, and used to push the cutter toward the proximal end of the elongated member.

Any appropriate size and/or shape of cutter may be used. For example, a tether cutter may be tubular or semitubular (e.g., having a shape similar to a half-pipe), or may have any other suitable configuration. As an example, a cutter may be in the form of a flat blade. Moreover, tether cutters may have any appropriate cutting edge configuration. For example, a cutter may have a beveled cutting edge, a sharpened outer cutting edge, or a sharpened inner cutting edge. In some variations, a cutter (e.g., a tubular cutter) may have a serrated or saw-tooth pattern of sharp protrusions around its perimeter to aid in cutting. Such variations may be used, for example, in cases in which the tubular cutter is spun or rotated during the cutting process. In certain variations, a cutter (e.g., a tubular cutter) may have a V-shaped cutting edge designed to channel a tether, and/or may have any other appropriate notched feature. A cutter may have a curved cutting edge, an angled cutting edge, a serrated cutting edge, a saw-tooth cutting edge, or any other suitable cutting edge. A serrated or saw-tooth cutting edge may be useful, for example, in a tubular cutter that is rotated or spun during the cutting process. Tubular cutters, as well as other types of cutters, can be configured such that they operate either externally or internally to a catheter.

A tether cutter may comprise any appropriate structure or material. In addition to the cutters described above, other examples of tether cutters include tether cutters that cut by heat, electricity, chemical reaction, or the like. For example, in some variations, a tether cutter may comprise an electrode or filament through which electrical energy is applied to cut a tether.

In some variations, multiple cutting devices may be used together to cut one or more tethers. The cutting devices may be the same as each other or different from each other. Moreover, while tether cutting has been described, in some variations, a tether may not be cut, or the tether may be cut, but may still maintain some length. The extra tether length may, for example, be used to help maintain the tether in a locked state (e.g., by preventing one or more knots in the tether from coming undone).

While locking devices and cutting devices have been described, in some variations, a single device may provide both locking and cutting functions. For example, a single device may provide both knot pushing and tether cutting functions.

While certain variations of locking devices, cutting devices, and related methods have been described above, other variations may be used. As an example, in some variations, one or more locking and/or cutting device components may include one or more radiopaque markers (e.g., platinum markers). Such markers may enhance visualization of a component and identification of its location during a procedure (e.g., under X-ray fluoroscopy). Moreover, some variations of devices may be used to provide a cinching effect with a tether. These devices may be used for any procedure where these functions (or combinations thereof) are desired. Locking, cutting, and cinching devices are described, for example, in U.S. patent application Ser. Nos. 11/232,190 (published as US 2006/0190030 A1), 11/270,034 (published as US 2006/0122633 A1), 11/583,627 (published as US 2008/0172035 A1), 12/577,044 (filed on Oct. 9, 2009), and 12/576,955 (filed on Oct. 9, 2009), all of which are hereby incorporated by reference in their entirety.

Kits are also described here. In some variations, the kits may include at least one tether-locking device and/or at least one tether-cutting device. In certain variations, the kits may include multiple different tether-locking devices, such as multiple different knot pushers (e.g., having different shapes, configurations, etc.). In some variations, the kits may include one or more anchor deployment devices. Anchor deployment devices and methods are described, for example, in U.S. patent application Ser. No. 12/657,422, filed on Jan. 19, 2010, which is hereby incorporated by reference in its entirety. Of course, instructions for use may also be provided with the kits.

While devices, methods and kits have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the appended claims.

What is claimed is:

1. A knot pusher for advancing a knot in a tether, the knot pusher comprising:
an elongated member;
a first coupling member slidably coupled to the elongated member;
a second coupling member fixedly coupled to the elongated member, wherein at least a portion of the second coupling member extends distally of the elongated member;
a first bearing; and
a second bearing distal of the first bearing,
wherein the first and second bearings are fixedly coupled to the first coupling member and slidably coupled to the second coupling member.

2. The knot pusher of claim 1, wherein at least one of the first and second coupling members comprises a wire.

3. The knot pusher of claim 1, wherein the elongated member comprises a first lumen, and the first coupling member is slidably disposed within at least a portion of the first lumen.

4. The knot pusher of claim 3, wherein the elongated member comprises a second lumen, and the second coupling member is fixedly disposed within a portion of the second lumen.

5. The knot pusher of claim 1, wherein the first bearing comprises a first tapered portion and the second bearing comprises a second tapered portion.

6. The knot pusher of claim 5, wherein the first and second bearings are slidably coupled to the second coupling member at the first and second tapered portions.

7. The knot pusher of claim 1, wherein the first tapered portion of the first bearing has a first aperture therethrough and the second tapered portion of the second bearing has a second aperture therethrough, and wherein the first coupling member passes through the first and second apertures.

8. The knot pusher of claim 1, further comprising a tubular member having a proximal end, a distal end, and a lumen therethrough, wherein the elongated member is disposed within the tubular member.

9. The knot pusher of claim 8, wherein the tubular member comprises a wall portion having a first aperture therethrough.

10. The knot pusher of claim 9, wherein the first and second bearings are disposed within the tubular member, and the first aperture is positioned adjacent the first bearing.

11. The knot pusher of claim 9, further comprising a hypotube having a second aperture therethrough, wherein the hypotube is disposed within the tubular member such that the second aperture is aligned with the first aperture.

12. The knot pusher of claim 1, further comprising a cutting member.

13. A method of tying a knot in a tether coupled to body tissue using a knot pusher comprising first and second bearings and a coupling member slidably coupled to the first and second bearings, the method comprising:
winding a portion of the tether around the first and second bearings to form an open knot in the tether;
advancing the knot pusher distally to advance the open knot distally;
withdrawing the coupling member from the first and second bearings; and
forming a closed knot from the open knot after the coupling member has been withdrawn from the first and second bearings.

14. The method of claim 13, wherein the method comprises pulling the tether to form the closed knot from the open knot.

15. The method of claim 13, wherein the first and second bearings are disposed in a lumen of a sheath comprising a wall portion having an aperture therethrough, and wherein the method further comprises passing the tether through the aperture.

16. The method of claim 13, wherein the body tissue comprises cardiac tissue.

17. The method of claim 16, wherein the cardiac tissue comprises heart valve tissue.

18. The method of claim 17, wherein the heart valve tissue comprises mitral valve tissue.

19. The method of claim 13, wherein the tether is coupled to a plurality of anchors that are coupled to the body tissue.

20. The method of claim 13, wherein the method comprises using a lasso to wind the portion of the tether around the first and second bearings to form the open knot in the tether.
